(19) United States
(12) Patent Application Publication
(10) Pub. No.: US 2024/0198320 A1
(43) Pub. Date: Jun. 20, 2024

(54) ZIF-8 ORGANOMETALLIC FRAME CATALYST COMPOSITE, AND CARBON DIOXIDE CONVERSION METHOD USING SAME

(71) Applicant: Korea University Research and Business Foundation, Seoul (KR)

(72) Inventors: Jung-Kyu CHOI, Seoul (KR); Kwan-Young LEE, Seoul (KR); Gi-Hoon LEE, Ansan-si (KR)

(73) Assignee: Korea University Research and Business Foundation, Seoul (KR)

(21) Appl. No.: 18/552,589

(22) PCT Filed: Oct. 31, 2022
(86) PCT No.: PCT/KR2022/016802

§ 371 Date: Sep. 26, 2023

(30) Foreign Application Priority Data
Nov. 1, 2021 (KR) 10-2021-0147657
Oct. 28, 2022 (KR) 10- 2022-0141432

(51) Int. Cl. B01J 31/16; B01J 35/61; C07D 317/40

(52) U.S. Cl. CPC B01J 31/1691; B01J 35/618; C07D 317/40; B01J 2231/4288; B01J 2531/26

(57) ABSTRACT

The present disclosure relates to a ZIF-8 metal-organic framework catalyst composite including: a ZIF-8 metal-organic framework in a metal-organic framework; and water molecules provided inside the ZIF-8 metal-organic framework.

### TECHNICAL FIELD

The present disclosure relates to a ZIF-8 metal-organic framework catalyst composite and a method for converting carbon dioxide using the same.

### BACKGROUND ART

Carbon dioxide is one of the main culprits of global warming, and not only studies on methods for reducing carbon dioxide, but also methods for using it in industry are being actively conducted. Among them, as a method of using carbon dioxide, there are methods of storing or converting carbon dioxide in various ways such as geologic storage, mineral storage, and chemical conversion.

A carbon dioxide cycloaddition reaction, one of the various chemical conversion methods, is a reaction capable of preparing a cyclic carbonate by adding carbon dioxide to the inside of an epoxy compound. At this time, various types of cyclic carbonates (chloropropene carbonate, propylene carbonate, styrene carbonate) may be prepared according to the type of alkyl group (chloromethyl group, methyl group, phenyl group) of the epoxy compound used, and the prepared cyclic carbonates have high use value in various industrial fields such as polar solvents, fuel additives, and battery electrolytes.

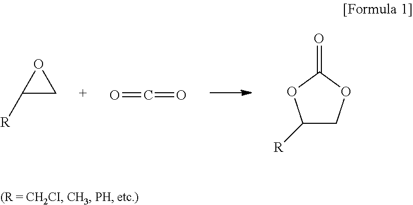

The carbon dioxide cycloaddition reaction (Formula 1) shows high reaction activity on a catalyst in which Lewis acids and bases coexist, and research is underway on the development of various types of catalysts to increase the efficiency of this reaction.

Further, metal-organic frameworks (MOFs), which are composed of bonds of metal cations and organic ligands, have high porosity, structural flexibility, and various functionalities so that they have high potential in various fields such as sensing, drug delivery, and catalysts. Zeolitic imidazolate framework-8 (ZIF-8), which is one of the MOFs, is composed of zinc (Zn) and 2-methylimidazolate, and may exhibit Lewis acids and bases simultaneously due to defects present on the external surface. Therefore, a cycloaddition reaction of various epoxy compounds and carbon dioxide may be promoted using ZIF-8.

### DISCLOSURE

### Technical Problem

An objective of the present disclosure is to provide a ZIF-8 metal-organic framework catalyst composite comprising water molecules.

Further, another objective of the present disclosure is to provide a method for promoting the conversion of carbon dioxide using the ZIF-8 metal-organic framework catalyst composite.

### Technical Solution

According to one aspect of the present disclosure, embodiments of the present disclosure may be a ZIF-8 metal-organic framework catalyst composite including: a ZIF-8 metal-organic framework in a metal-organic framework; and water molecules provided inside the ZIF-8 metal-organic framework.

In one embodiment, the water molecules may dissociate at least a portion of the bond of the ZIF-8 metal-organic framework to form a reaction site, and the ZIF-8 metal-organic framework catalyst composite may include a ZIF-8 metal-organic framework in which a reaction site is formed and a ZIF-8 metal-organic framework in which a reaction site is not formed.

In one embodiment, the reaction site may include: a first reaction site including a Zn—OH bond; and a second reaction site including an N—H bond.

In one embodiment, the formation ratio of the ZIF-8 metal-organic framework in which a reaction site is formed may be formed at a weight ratio of 0.05 to 1.0 as the ZIF-8 metal-organic framework in which a reaction site is formed/the ZIF-8 metal-organic framework in which a reaction site is not formed.

In one embodiment, at least one of the first reaction site and the second reaction site may form carbonate by promoting a cycloaddition reaction between carbon dioxide and an epoxide-based compound.

In one embodiment, the epoxide-based compound may be at least one of epichlorohydrin, ethylene oxide, styrene oxide, and propylene oxide.

In one embodiment, the yield of carbonate may increase as the content of water molecules increases.

In one embodiment, carbonate may be a cyclic carbonate, and may be at least one of chloropropene carbonate, ethylene carbonate, styrene carbonate, and propylene carbonate.

In one embodiment, the yield of carbonate may be calculated by Equation 2 below.

$$Yield of carbonate ( \% ) = \frac{Number of moles of formed carbonate}{Number of moles of supplied epoxide - based compound} \times 100 \qquad [ Equation 2 ]$$

In one embodiment, the yield of carbonate by the cycloaddition reaction may be 20% to 99%.

In one embodiment, the water molecules may be included at a weight ratio of 0.005 to 0.35 with respect to the ZIF-based metal-organic framework catalyst composite.

In one embodiment, the BET surface area of the ZIF-8 metal-organic framework may be 1,300 m²/g to 1,600 m²/g.

In one embodiment, the ZIF-8 metal-organic framework in which a reaction site is formed may have a peak for binding energy indicating the formation of Zn—OH in X-ray photoelectron spectroscopy (XPS) analysis, representing a first peak at 1,022.3 eV to 1,022.5 eV and a second peak at 531.8 eV to 520 eV.

In one embodiment, in X-ray photoelectron spectroscopy (XPS) analysis, the ratio of an XPS peak area of the ZIF-8 metal-organic framework in which a reaction site is formed to that of the ZIF-8 metal-organic framework representing the binding energy of Zn—N may be 0.4 to 0.8.

Furthermore, according to one aspect of the present disclosure, embodiments of the present disclosure may be related to a method for converting carbon dioxide using a ZIF-8 metal-organic framework catalyst composite, as a method for converting carbon dioxide using a ZIF-8 metal-organic framework catalyst composite having at least one of the above-described characteristics, the method including the steps of: putting a material containing the ZIF-8 metal-organic framework catalyst composite and an epoxide-based compound into an autoclave; supplying carbon dioxide into the autoclave; heating the inside of the autoclave to obtain carbonate by reacting the ZIF-8 metal-organic framework catalyst composite, the epoxide-based compound, and carbon dioxide; and quenching the product.

In one embodiment, the inside of the autoclave may be heated to 40° C. to 200° C.

In one embodiment, carbon dioxide may be supplied until it reaches a pressure of 1 bar to 30 bar.

In one embodiment, the yield of carbonate may be calculated by Equation 2 below.

$$Yield of carbonate ( \% ) = \frac{Number of moles of formed carbonate}{Number of moles of supplied epoxide - based compound} \times 100 \qquad [ Equation 2 ]$$

In one embodiment, the yield of carbonate may be 20% to 99%.

### Advantageous Effects

According to the present disclosure as described above, it is possible to provide a ZIF-8 metal-organic framework catalyst composite that promotes the conversion reaction of carbon dioxide, and a method for promoting carbon dioxide conversion using the same.

### DESCRIPTION OF DRAWINGS

FIG. 1 is a schematic diagram showing a process in which a Zn—N bond is dissociated (hydrolyzed) in a ZIF-8 metal-organic framework catalyst composite while a cycloaddition reaction of carbon dioxide is taking place in one embodiment according to the present disclosure.

FIG. 2A shows TGA profiles of ZIF-8W and ZIF-8W_T24_Im, and FIG. 2B shows water vapor adsorption isotherms of ZIF-8W-T24 and ZIF-8x (x=M and C) at 70° C.

FIGS. 3A and 3B show N₂ physical adsorption isotherms of ZIF-8W and ZIF-8M degassed at 80° C. and 200° C. for 4 hours, respectively.

FIG. 4 shows XPS analysis results for ZIF-8W, ZIF-8W-S, and DP.

FIG. 5 shows XPS spectra at N is and Zn 2p of ZIF-8x and ZIF-x_S (x=W, M, and C).

FIG. 6 shows XAES spectroscopy of Zn LMMs for fresh ZIF-8x and spent ZIF-8x_S (x=W, M, and C).

FIGS. 7 to 9 show yields, external surface compositions, electron microscope images, and XRD patterns for ZIF-8W, M, and C.

FIG. 10 shows size distributions of respective catalyst particles.

FIG. 11 shows SEM images for ZIF-8W and ZIF-8W_S.

FIG. 12 shows XRD patterns of ZIF-8W, ZIF-8W_S, and DP according to the simulated XRD patterns of ZIF-8.

FIG. 13 shows results of performing TGA and FT-IR analysis on ZIF-8W, ZIF-8M, and ZIF-8C.

FIG. 14 shows results of experiments conducted by thermally treating ZIF-8W to remove water molecules or mixing with DP in order to confirm the catalytic properties of ZIF-8W.

FIG. 15 shows XRD patterns measured by mixing ZIF-8W or DP with α-alumina at various ratios in order to find out the state of ZIF-8W having catalytic activity for carbon dioxide cycloaddition reaction.

FIG. 16 is a graph in which the weight ratio of a ZIF-8 intermediate derivative is estimated from the weight ratio of ZIF-8 and DP in the catalyst after reaction, and shows the weight ratio and the CC yield of the ZIF-8 intermediate derivative according to the reaction time.

FIG. 17 shows TGA results of thermally treated ZIF-8W.

FIG. 18 shows results of measuring N₂ adsorption desorption isotherms, pore distributions, and FT-IR of respective samples in order to find out the site acting as a catalyst in ZIF-8W.

FIG. 19 shows N₂ adsorption-desorption isotherms and micropore size distributions on the logarithmic x-axis for ZIF-8 samples.

FIG. 20 shows size distributions of mesopores for ZIF-8 samples.

FIG. 21 shows FT-IR spectra for ZIF-8 samples.

FIG. 22 is XPS results for O 1s for ZIF-8 samples.

FIG. 23 is ones examining the compositions of elements on the surface of ZIF-8 samples.

FIG. 24 shows XRD patterns, O 1s XPS spectra, N₂ adsorption-desorption isotherms, micropore size distributions, and NH₃ TPD profiles of ZIF-8W samples.

FIG. 25 shows XPS spectra in N 1s and Zn 2p for ZIF-8 samples, and external surface compositions.

FIG. 26 shows CO₂ and NH₃ temperature programmed desorption (TPD) profiles for ZIF-8 samples.

FIG. 27 shows TPD profiles of ZIF-8 samples, FT-IR spectra of ZIF-8 samples after NH₃ adsorption experiments, and FT-IR spectra of samples after NH₃ adsorption experiments and thermal treatment at different temperatures.

FIG. 28 shows FT-IR spectra of ZIF-8 samples before and after NH₃ adsorption experiments.

FIG. 29 shows information on deconvolution curves of ZIF-8 samples.

### MODES OF THE INVENTION

Details of other embodiments are included in the detailed description a nd drawings.

Advantages and features of the present disclosure, and methods of achieving them, will become clear with reference to the detailed description of the following embodiments taken in conjunction with the accompanying drawings.

However, the present disclosure is not limited to the embodiments disclosed below, but may be implemented in various different forms, and since, unless otherwise specified in the following description, these numbers are essentially approximations that reflect the various uncertainties of measurement that arise in obtaining these values among other things, it should be understood that all numbers, values and/or expressions expressing components, reaction conditions, or amounts of the components in the present disclosure are in all instances modified by the term “about”. Also, when a numerical range is disclosed in the present description, such a range is continuous and includes all values in such a range from a minimum value to the maximum value including a maximum value, unless otherwise indicated. Furthermore, when such a range refers to an integer, all integers including from the minimum value to the maximum value including a maximum value are included unless otherwise indicated.

Further, when a range is stated for a variable in the present disclosure, it will be understood that the variable includes all values within the stated range including the stated endpoints of the range. For example, a range of “5 to 10” includes any subrange of 6 to 10, 7 to 10, 6 to 9, 7 to 9, and the like as well as values of 5, 6, 7, 8, 9, and 10, and it will be understood also to include any value between integers that fall within the scope of the stated range, such as 5.5, 6.5, 7.5, 5.5 to 8.5, 6.5 to 9, and the like. For example, a range of “10% to 30%” includes any subrange of 10% to 15%, 12% to 18%, 20% to 30%, and the like as well as values such as 10%, 11%, 12%, 13% and the like, and all integers including up to 30%, and it will be understood also to include any value between integers that fall within the scope of the stated range, such as 10.5%, 15.5%, 25.5%, and the like.

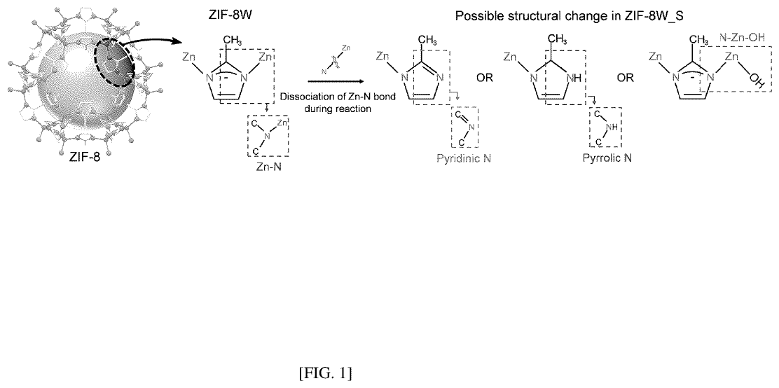

FIG. 1 is a schematic diagram showing a process in which a Zn—N bond is dissociated (hydrolyzed) in a ZIF-8 metal-organic framework catalyst composite while a cycloaddition reaction of carbon dioxide is taking place in one embodiment according to the present disclosure. It shows that the Zn—N bonds on the external surface of the ZIF-8 metal-organic framework may be dissociated and converted into pyrrolic N, pyridinic N, and N—Zn—OH structures. Among them, the Zn—OH structure may be spent as a catalyst by providing a Lewis acid/base moiety in the cycloaddition reaction of carbon dioxide.

The ZIF-8 metal-organic framework catalyst composite according to one embodiment of the present disclosure may include, in the metal-organic framework, a ZIF-8 metal-organic framework; and water molecules provided inside the ZIF-8 metal-organic framework.

The catalyst composite according to one embodiment of the present disclosure may include a reaction site on the external surface of the ZIF-8 metal-organic framework, and may have a catalytic activity that promotes the reaction of CO₂ and an epoxide compound by the reaction site.

The water molecules existing inside the ZIF-8 metal-organic framework may dissociate at least a portion of the bond of the ZIF-8 metal-organic framework to form a reaction site. Here, the water molecules may be expressed as being occluded inside the ZIF-8 metal-organic framework.

The water molecules may exit the ZIF-8 metal-organic framework structure while hydrolyzing (dissociating) a Zn—N bond among the N—Zn—N bonds present in the ZIF-8 metal-organic framework structure. In this process, the Zn—N bond may be divided into a Zn—OH bond and an N—H bond and form a reaction site. Accordingly, the reaction site may include at least one of a first reaction site including a Zn—OH bond and a second reaction site including an N—H bond.

The water molecules may hydrolyze at least a portion of N—Zn—N present in the ZIF-8 metal-organic framework. As a result, the formation ratio of the ZIF-8 metal-organic framework in which the reactive site produced is formed may be formed at a weight ratio of 0.05 to 1.0 as a ZIF-8 metal-organic framework in which the reactive site is formed/a ZIF-8 metal-organic framework in which the reaction site is not formed. Alternatively, the ZIF-8 metal-organic framework on which the reactive site is formed/the ZIF-8 metal-organic framework on which the reactive site is not formed may be formed at a weight ratio of 0.05 to 0.9. Alternatively, the ZIF-8 metal-organic framework on which the reactive site is formed/the ZIF-8 metal-organic framework on which the reactive site is not formed may be formed at a weight ratio of 0.05 to 0.69. When the ratio of the ZIF-8 metal-organic framework on which the reactive site is formed/the ZIF-8 metal-organic framework on which the reactive site is not formed is less than 0.05, the catalytic activity for the cycloaddition reaction may not be sufficient.

At least one of the first reaction site and the second reaction site may promote the cycloaddition reaction between carbon dioxide and an epoxide-based compound to efficiently form carbonate. The first reaction site may simultaneously act as a Lewis acid and a Lewis base in the cycloaddition reaction. Specifically, the Zn moiety in the first reaction site may act as a Lewis acid, and the OH moiety may act as a Lewis base.

The water molecules may be included at a weight ratio of 0.005 to 0.35 with respect to the ZIF-based metal-organic framework catalyst composite. The water molecules may exist inside the pore structure of the ZIF-based metal-organic framework. Therefore, the above-described range may present in a minimum amount in which the water molecules can cause dissociation of N—Zn—N bonds that are in the ZIF-based metal-organic framework or present in a maximum amount that can exist inside the ZIF-based metal-organic framework.

In this case, the yield of carbonate may increase as the content of the water molecules present in the ZIF-8 metal-organic framework increases. As the content of the water molecules increases, more N—Zn—N bonds of the ZIF-8 metal-organic framework may be dissociated, and thus catalytic activity may be increased. For example, as the content of water molecules decreases to 8 wt %, 4.2 wt %, 2.1 wt %, 0.5 wt %, and 0.1 wt %, the yield of carbonate may be decreased to 20.5%, 9.9%, 4.5%, and 2.6%, respectively.

As a result of the cycloaddition reaction promoted by the ZIF-8 metal-organic framework, the yield of carbonate may be calculated by Equation 2 below.

$$Yield of carbonate ( \% ) = \frac{Number of moles of formed carbonate}{Number of moles of supplied epoxide - based compound} \times 100 \qquad [ Equation 2 ]$$

The yield of carbonate according to Equation 2 may be 20% to 99%. In addition, the yield of carbonate according to Equation 2 may be 40% to 60%.

In the cycloaddition reaction, the epoxide-based compound may be used without limitation as long as it includes an epoxide structure. For example, the epoxide-based compound may be at least one of epichlorohydrin, ethylene oxide, styrene oxide, and propylene oxide.

Further, carbonate in the cycloaddition reaction may be formed depending on the type of an epoxide-based compound used. For example, carbonate may be a cyclic carbonate. Also, for example, the cyclic carbonate may be at least one of chloropropene carbonate, ethylene carbonate, styrene carbonate, and propylene carbonate.

The ZIF-8 metal-organic framework may have a BET surface area of 1,300 m²/g to 1,600 m²/g.

The peaks for binding energy indicating the formation of Zn—OH in X-ray photoelectron spectroscopy (XPS) analysis of at least a portion of the ZIF-8 metal-organic framework catalyst composite may indicate a first peak at 1,022.3 eV to 1,022.5 eV and a second peak at 531.8 eV to 520 eV.

Further, in the X-ray photoelectron spectroscopy (XPS) analysis of the ZIF-8 metal-organic framework catalyst composite, the ratio of the XPS peak area of the ZIF-8 metal-organic framework including the reactive site to the XPS peak area of the ZIF-8 metal-organic framework representing the binding energy of Zn—N may be 0.4 to 0.8.

According to another aspect of the present disclosure, the embodiments of the present disclosure may include a method for converting carbon dioxide using a ZIF-8 metal-organic framework catalyst composite, as a method for converting carbon dioxide using a ZIF-8 metal-organic framework catalyst composite including at least one of the above-described characteristics, the method including the steps of: putting a material containing the ZIF-8 metal-organic framework catalyst composite and an epoxide-based compound into an autoclave; supplying carbon dioxide into the autoclave; heating the inside of the autoclave to obtain a product by reacting the material containing the ZIF-8 metal-organic framework catalyst composite, the epoxide-based compound, and carbon dioxide; and quenching the product to obtain carbonate.

The inside of the autoclave may be heated to 40° C. to 200° C. Alternatively, the inside of the autoclave may be heated to 60° C. to 80° C. When the heating temperature of the inside of the autoclave is less than 40° C., the reaction between carbon dioxide and the epoxide-based compound may not appropriately occur since the temperature is too low. In addition, when the heating temperature of the inside of the autoclave exceeds 200° C., compounds may be thermally decomposed and a desired reaction may not occur.

Carbon dioxide supplied to the autoclave may be used to form carbonate by reacting with the epoxide-based compound. Carbon dioxide may be supplied until it reaches a pressure of 1 bar to 30 bar. In addition, carbon dioxide may be supplied until it reaches a pressure of 7 bar to 9 bar. The pressure of carbon dioxide in the autoclave may be maintained while the reaction proceeds. When the pressure of carbon dioxide is less than 1 bar, the cycloaddition reaction may not appropriately occur due to the small amount of carbon dioxide. In addition, when the pressure of carbon dioxide exceeds 30 bar, a side reaction may occur and the yield of carbonate may decrease.

The yield of carbonate may be calculated by Equation 2 below.

$$Yield of carbonate ( \% ) = \frac{Number of moles of formed carbonate}{Number of moles of supplied epoxide - based compound} \times 100 \qquad [ Equation 2 ]$$

Further, the yield of carbonate may be calculated by multiplying the conversion rate of the epoxide-based compound by the selectivity of carbonate. Here, the selectivity of carbonate may be calculated as 100% since there is no side reaction product. At this time, the conversion rate (%) of the epoxide-based compound may be calculated by Equation 3.

$$Conversion rate of epoxide ‐ based compound ( \% ) = \frac{Number of moles of reacted epoxide - based compound}{Number of moles of supplied epoxide - based compound} \times 100 \qquad [ Equation 3 ]$$

The yield of carbonate calculated by Equation 2 may be a ratio of carbonate obtained by reacting at least one of carbon dioxide and an epoxide-based compound, which is a reactant, until it is entirely consumed. Therefore, the yield of carbonate may be 100% when both of carbon dioxide and the epoxide-based compound are sufficiently present. The yield of carbonate calculated by Equation 2 above may be 20% to 99%. In addition, the yield of carbonate calculated by Equation 2 above may be 40% to 60%.

Example and Comparative Examples of the present disclosure are described below. However, the following Examples are only preferred embodiments of the present disclosure, and the scope of rights of the present disclosure is not limited by the following Examples.

Terms in this specification and drawings are as follows. ZIF-8 may be a concept that includes general ZIF-8 and all types of ZIF-8 spent herein. ZIF-8W may refer to one synthesized using water as a solvent when synthesizing ZIF-8. ZIF-8W_S may refer to a state after using ZIF-8W as a catalyst. Fresh ZIF-8W may refer to a state before a Zn—N decomposition reaction by water proceeds as a state immediately after synthesis. ZIF-8M may refer to one synthesized using methanol as a solvent during synthesis. ZIF-8C may refer to a commercial product. DP may refer to a new dense phase generated from ZIF-8. ZIF-8W_Tx may refer to one obtained by removing water from fresh ZIF-8W. x may refer to a thermal treatment time to remove water. In addition to this, terms used in the drawings may be defined in the following description.

All chemicals used in the following Examples and Experimental Examples were used as they were without further purification.

### Preparation Example

In order to synthesize ZIF-8 containing water inside the structure of ZIF-8, a zinc precursor and an aqueous 2-methylimidazole solution were prepared, respectively. A zinc precursor solution was prepared by dissolving 1.7 g of zinc nitrate (Zn(NO₃)₃·6H₂O, 98%, Sigma-Aldrich; product number: 228737) in 18 mL of deionized (DI) water. A 2-methylimidazole solution was prepared by dissolving 2-methylimidazole (22.7 g, 99%, Sigma-Aldrich; product number: M50850) in 70 mL of DI water. Subsequently, the zinc precursor solution was added to the 2-methylimidazole solution. The mixture was reacted while performing stirring at room temperature for about 20 minutes. Thereafter, a cycle of centrifugation, decanting, and washing with DI water three times was performed, and finally dried at 70° C. for at least 12 hours to recover a solid product.

### Example

ZIF-8W, which is ZIF-8 containing water molecules, was synthesized by the synthesis method of Preparation Example.

### Comparative Examples

### 1. Preparation of Thermally Treated ZIF-8W

In addition to fresh ZIF-8W, the synthesized ZIF-8W was thermally treated at 100° C. for various times (6, 12, 18, and 24 hours) to obtain thermally treated ZIF-8W. For convenience, the thermally treated ZIF-8W is denoted as ZIF-W_Ty, where y represents the thermal treatment duration (h).

TABLE 1

| | Denotation | Thermal treatment time |
|---|---|---|
| Comparative | ZIF-W_T6 | 6 hours |
| Example 1-1 | | |
| Comparative | ZIF-W_T12 | 12 hours |
| Example 1-2 | | |
| Comparative | ZIF-W_T18 | 18 hours |
| Example 1-3 | | |
| Comparative | ZIF-W_T24 | 24 hours |
| Example 1-4 | | |

### 2. Synthesis of ZIF-8M

In order to synthesize ZIF-8M containing methanol inside the structure of ZIF-8, a zinc precursor and a 2-methylimidazole solution were prepared, respectively. Zinc nitrate (2.93 g, 9.87 mmol) and 2-methylimidazole (6.49 g, 79.0 mmol) were each dissolved in 200 mL of methanol (99.8%, Sigma-Aldrich; product number: 322415). The two solutions were mixed and allowed to react while performing stirring at room temperature for about 2 hours. Thereafter, a solid product was recovered by performing centrifugation, decanting, and washing with methanol three times, and finally performing drying at 70° C. for at least 12 hours.

### 3. For ZIF-8C, Commercially Available ZIF-8 was Purchased and Spent.

### 4. DP (Dense Phase) Synthesis

A DP (dense phase) was synthesized in order to compare the reaction of ZIF-8W in addition to the ZIF-8 phase. To this end, zinc nitrate (2.1 g, 7.06 mmol) and 2-methylimidazole (0.6 g, 7.21 mmol) were dissolved in 180 mL of dimethylformamide (DMF, 99%, Sigma-Aldrich; product number: 227056). 100 mL of DI water was added to this mixture. The mixture was stirred until it reached near room temperature and then transferred to a Teflon liner. The reaction was performed at 140° C. for about one day while rotating (about 60 rpm) a sealed Teflon-lined autoclave. Next, the reaction was quenched using tap water. A product was recovered through a cycle of centrifugation, decanting, and washing with DMF three times, and finally dried at 70° C. for at least 12 hours. For convenience, the recovered product is named DP, which indicates that it has a dense phase.

### [Test Method]

### 1. Performing CO₂ Cycloaddition Reaction

The CO₂ cycloaddition reaction on the ZIF-8 catalyst was carried out in a specially fabricated Teflon-lined stainless-steel autoclave (internal volume of Teflon-liner: 250 mL). ZIF-8 (0.72 g) and epichlorohydrin (ECH, 10 mL, 99%, Sigma-Aldrich; product number: 45340) were put into a Teflon liner. Thereafter, the Teflon liner containing the reactants and catalyst was positioned inside the autoclave. Next, CO₂ gas was filled in the autoclave until a pressure of 7 bar was reached, and the reactor was heated to a desired reaction temperature (70° C.) using an electric heating jacket. For consistency, the reaction time was measured after the temperature of the reactor had reached the reaction temperature and the reaction was performed for about 4 hours. After the specified time, the reaction was quenched with tap water. After that, residual gas was released, and the spent catalyst and liquid product were centrifuged, respectively. The spent ZIF-8 catalyst was recovered by performing a cycle of centrifugation, decanting, and washing with DI water three times, and performing drying at 70° C. for at least 12 hours. For convenience, spent catalyst is denoted ZIF-8x_S, where x represents M, W, or C from those corresponding to fresh catalyst (ZIF-8x), and S points to the spent catalyst. The obtained liquid product was analyzed using a gas chromatograph (YL6500, Young In Chromass, South Korea) equipped with a flame ionization detector and a capillary column (DB-5, 30×0.25 mm, Agilent). Toluene was used as an internal standard to ensure an accurate measurement of catalytic activity. Chloropropene carbonate (CC) was measured by multiplying the conversion rate of ECH by the selectivity of CC.

The selectivity of CC was assumed to be 100% since there were no other products including chloro-1,2-propanediol (diol) and 2,5-bis(chloromethyl)-1,4-dioxane (dimer) in both liquid- and gas-phase products. Equation 2 below is an equation used to calculate the yield of chloropropene carbonate, and may be equally applied to other carbonates. In addition, Equation 3 was used to calculate the conversion rate of epichlorohydrin and may be equally applied to other epoxide-based compounds.

$$Yield of carbonate ( \% ) = \frac{Number of moles of formed carbonate}{Number of moles of supplied epoxide - based compound} \times 100 \qquad [ Equation 2 ]$$

$$Conversion rate of epoxide ‐ based compound ( \% ) = \frac{Number of moles of reacted epoxide - based compound}{Number of moles of supplied epoxide - based compound} \times 100 \qquad [ Equation 3 ]$$

Experiments with different reaction times and catalyst weights in addition to the above CO₂ cycloaddition reaction conditions (standard conditions) were additionally performed in the case of ZIF-8W. Specifically, the reaction performed with 0.72 g of ZIF-8W was carried out by setting the reaction duration to 1, 2, 3, 6, 10, and 16 hours, respectively, and the weight of ZIF-8W was varied while increasing it in increments of 0.18 g from 1 g to 0.54 g with a reaction time of 4 hours. In addition, the mixture of ZIF-8W and DP having a total mass of catalyst of 0.72 under standard conditions was set and performed to a weight (g) ratio of ZIF-8/DP mixture of 0/0.72, 0.18/0.54, 0.36/0.36, and 0.54/0.18. Finally, the reaction of completely dried ZIF-8W (i.e., ZIF-8W_T24) was carried out under reaction conditions including water, where the ratio of water added to ZIF-8W_T24 (0.72 g) was 8 wt % (the same as water adsorbed in the synthesized ZIF-8W), 20 wt %, and 40 wt % of the catalyst. In addition, for convenience, ZIF-8W_T24 used in reaction conditions including water (8 wt %) is indicated as ZIF-8W_T24_S_H, where the character H is attached to indicate humid reaction conditions.

### 2. Property Analysis

To confirm the structure of ZIF-8, X-ray diffraction patterns of ZIF-8 samples were obtained using a Rigaku Model D/MAX-2500V/PC (Japan) equipped with a RINT2000 vertical goniometer (40 kV, 100 mA and λ=1.54 Å). The crystallographic information file (CIF) for ZIF-8 was downloaded from the Cambridge Crystallographic Data Centre website (CCDC, www.ccdc.cam.ac.uk: Deposition No. 602542), and Mercury software (also available on the CCDC website) for generating XRD patterns of simulated ZIF-8 was used. For reliable XRD analysis, α-alumina powder and ZIF-8x or ZIF-8x_S (x=W, M, and C) were physically mixed at equal weight ratios (w/w=1) and used as internal standards. XRD analysis was also used to evaluate the ratio of possible active components as a function of reaction time. First, XRD patterns of pure ZIF-8 or DP particles mixed with α-alumina powder at various weight ratios (0.1, 0.5, and 1) were obtained. Next, a good linear correlation between the weight ratio and the peak area ratio of the samples was obtained for both of ZIF-8 and DP by being provided as a calibration curve. Thereafter, the ratio or phase (not both ZIF-8 and DP) of the intermediate derivative could be measured indirectly by tracking the ratio of ZIF-8 and DP of 100%.

Hitachi S-4800 field emission scanning electron microscope (SEM, Japan) and FEI Tecnai G² F30ST field-emission transmission electron microscope (TEM, USA) were used to obtain SEM and TEM images of ZIF-8x and ZIF-8x_S (x=W, M, and C), respectively. The N₂ adsorption desorption isotherms of ZIF-8x, ZIF-8x_S (x=W, M, and C) and examples were obtained at 77 K using an ASAP 2020 (Micromeritics Inc., USA), and all samples were subjected to degassing at 80° C. for about 4 hours under vacuum prior to measurement. Thereafter, such isotherms were each used to calculate the specific surface area through the Brunauer-Emmett-Teller (BET) equation and calculate the micropore and mesopore size distributions using the Horvath-Kawazoe (H-K) and Barrett-Joyner-Halenda (BJH) methods. Thermogravimetric analysis (TGA) was performed to measure the content of water in ZIF-8W using a Q50 instrument (TA Instruments, USA). In the TGA, the powder samples were heated from room temperature at a ramp rate of 5° C.·min⁻¹ under nitrogen having a flow of 100 mL·min⁻¹. To confirm whether the process of adsorbing water on ZIF-8 is reversible or irreversible, two experiments (experiments on water in liquid and gaseous states) were performed using a fully dried ZIF-8W (ZIF-8W_T24). First, regarding the adsorption of water in liquid state, ZIF-8W_T24 was immersed in water at 25° C. for 1 day while keeping the weight ratio of ZIF-8 to water at 0.1. The process of centrifuging ZIF-8W_T24 immersed in water (denoted by ZIF-8W_T24_Im, where Im represents water immersion) and drying it at 70° C. for at least 12 hours to collect it was conducted, as done for as-synthesized ZIF-8x (x=W and M). TGA was performed on the immersed ZIF-8W-T24. In addition, the water adsorption isotherms of ZIF-8W_T24 (also ZIF-8M and ZIF-8C) were measured at 70° C., which was same as the reaction temperature, using a vapor sorption analyzer (DVS Vacuum, Surface Measurement System, UK).

Further, in order to evaluate acido-basicities of ZIF-8x, ZIF-8x_S (x=W, M, and C) and DP, temperature-programmed desorption analysis (TPD analysis) using various probe molecules (basic CO2 and acidic NH3) was performed using BELCAT II (MicrotracBEL Corp., Japan). In order to remove adsorbed molecules, ZIF-8x, ZIF-8x_S (x=W, M, and C) and examples were heated at 300° C. for 1 hour under a helium flow prior to TPD measurement, and subsequently exposed to a CO₂ or NH₃ at 0° C. for 1 hour. After sufficient adsorption was performed, the samples were heated from 0° C. to 300° C. at a ramping rate of 10° C.·min⁻¹ under a helium flow of 30 mL·min⁻¹. Molecules desorbed during heating were detected online using a thermal conductivity detector (TCD).

The chemical compositions of ZIF-8x, ZIF-8x_S (x=W, M, and C), and DP were performed by Fourier transform infrared (FT-IR) spectroscopy, and this was performed using a Nicolet™ iS50 FT-IR spectrometer (Thermo Scientific™, USA). Specifically, ZIF-8W, ZIF-8W_S, DP, and 2-methylimidazole pellets prepared by pressing the sample mixture with KBr were put in a specially fabricated in-situ cell equipped with KBr windows on both sides thereof. KBr pellets deposited with ZIF-8 were thermally treated at 150° C. for about 120 minutes under vacuum, and subsequently, cooled to room temperature. Finally, chemical compositions not only for ZIF-8x, ZIF-8x-S (x=W, M, and C), and DP, but also for ZIF-8x, ZIF-8x-S (x=W, M, and C), and DP that were adsorbed with NH₃ were examined using the attenuated total reflection (ATR) mode. For FT-IR analysis of NH₃-adsorbed samples, the samples were prepared using BELCAT II. The samples were heated at 150° C. for 1 hour under a helium flow in order to remove adsorbed molecules, and subsequently exposed to NH₃ at 30° C. for 1 hour. After sufficient adsorption water performed, the NH₃-adsorbed samples were heated from 30° C. to x° C. at a ramping rate of 10° C.·min⁻¹ under a helium flow of 30 mL·min⁻¹ (\textcircled{1} x=50, 100, and 150° C. for ZIF-8W, ZIF-8W_S, and DP, \textcircled{2} x=50° C. for ZIF-8M_S and ZIF-8C_S). Such adsorption process was carried out by varying the amount of NH₃. Finally, X-ray photoelectron spectroscopy (XPS) and X-ray excited Auger electron spectroscopy (XAES) were performed using an X-tool (Ulvac-PHI, Japan) equipped with a monochromatic Al Kα X-ray source (hv=1,486.6 eV, 15 kV, and 24.1 W) in order to detect the species of Zn, N, and O in the ZIF-8x, ZIF-8x-S (x=W, M, and C) and DP. All measured binding energies were referenced to the C is peak at 284.5 eV. For reliable deconvolution of FT-IR and XPX spectra, the software of Magic Plots Student 2.5.1 was used.

### Experiment Results

### 1. Property Analysis of ZIF-8

### (1) Water Adsorption Characteristics of ZIF-8

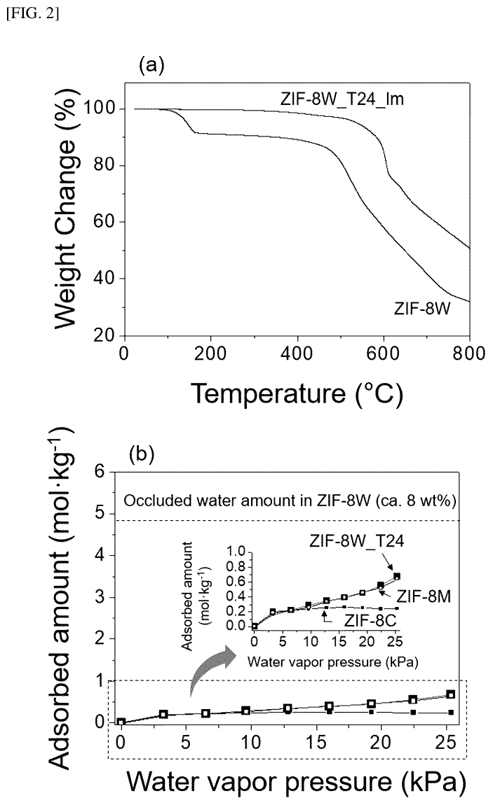

FIG. 2A shows TGA profiles of ZIF-8W and ZIF-8W_T24_Im, and FIG. 2B shows water vapor adsorption isotherms of ZIF-8W-T24 and ZIF-8x (x=M and C) at 70° C. In FIG. 2B, the area inside the dotted line box is enlarged and inserted to be indicated, and for comparison, the amount of water occluded in ZIF-8W is indicated by a dotted line below 5 on the y-axis. The TGA profile of ZIF-8W_T24 (ZIF-8W_T2_Im) immersed in water for 1 day showed negligible water adsorption properties (FIG. 2A). In addition, the water adsorption isotherm of ZIF-8W_T24 at 70° C. shows a very low H₂O adsorption capacity (FIG. 2B), and this is consistent with the previously reported content. In the result like this as described later, the water molecules occluded in the synthesized ZIF-8W are important in generating active sites for a CO₂ cycloaddition reaction, and the result strongly supports the fact that the fully dried ZIF-8 does not form the active sites for the CO₂ cycloaddition reaction since the water molecules cannot be adsorbed onto fully dried ZIF-8 at intermediate pressures.

### (2) Textural Properties of ZIF-8

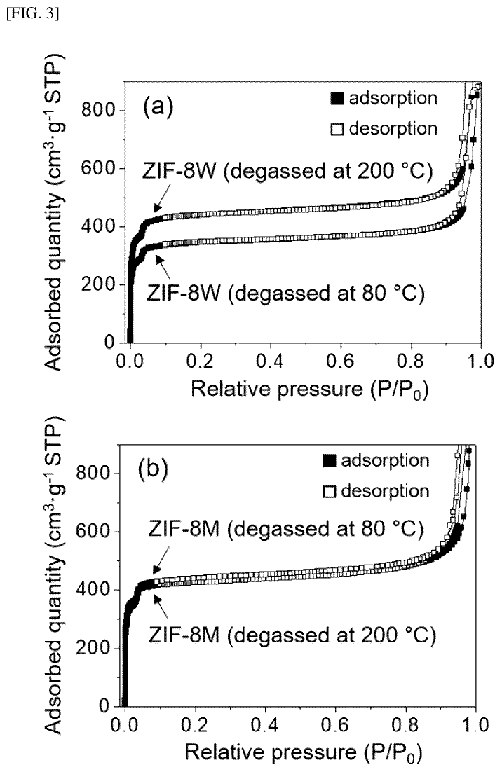

FIGS. 3A and 3B show N₂ physical adsorption isotherms of ZIF-8W and ZIF-8M degassed at 80° C. and 200° C. for 4 hours, respectively. Table 2 shows N₂ physical adsorption quantities and BET surface areas corresponding thereto. The physical adsorption isotherm data in Table 2 were calculated using the Brunauer-Emmett-Teller equation. Three types of ZFI-8 were degassed at 80° C. for N₂ physical adsorption, and the water molecules occluded inside ZIF-8W remained to some extent even after degassing. Nevertheless, steps of change inherent to ZIF-8W were observed. A degassing temperature similar to the reaction temperature (70° C.) of the cycloaddition reaction was used to avoid undesirable variation of the textural properties due to the conventionally higher degassing temperature, whereas it was applied in examining the change (particularly, the inherent step change) in the textural properties of ZIF-8 before and after the reaction. When both of ZIF-8W and ZIF-8M were degassed at a higher temperature (at 200° C. rather than 80° C.), their N2 physical adsorption quantities and BET surface areas corresponding thereto were comparable to each other, and this indicates that water molecules in ZIF-8W were completely removed.

TABLE 2

| | Degassing temperature | |
|---|---|---|
| Catalyst | (° C.) | S_{BET}^{a} (m² · g⁻¹) |
| ZIF-8W | 80 | 1,390 ± 11 |
| | 200 | 1,530 ± 13 |
| ZIF-8M | 80 | 1,592 ± 2 |
| | 200 | 1,568 ± 4 |

### (3) Chemical Species Analysis on the External Surfaces of ZIF-8 and DP

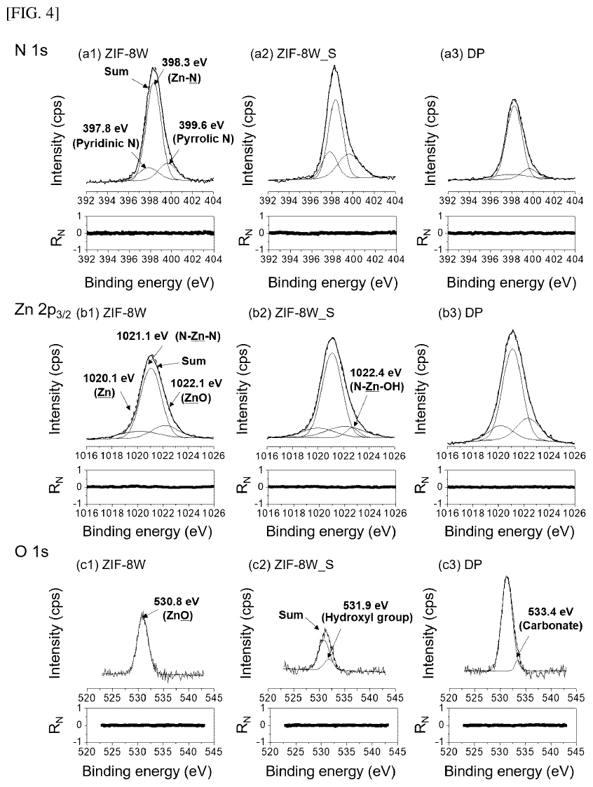

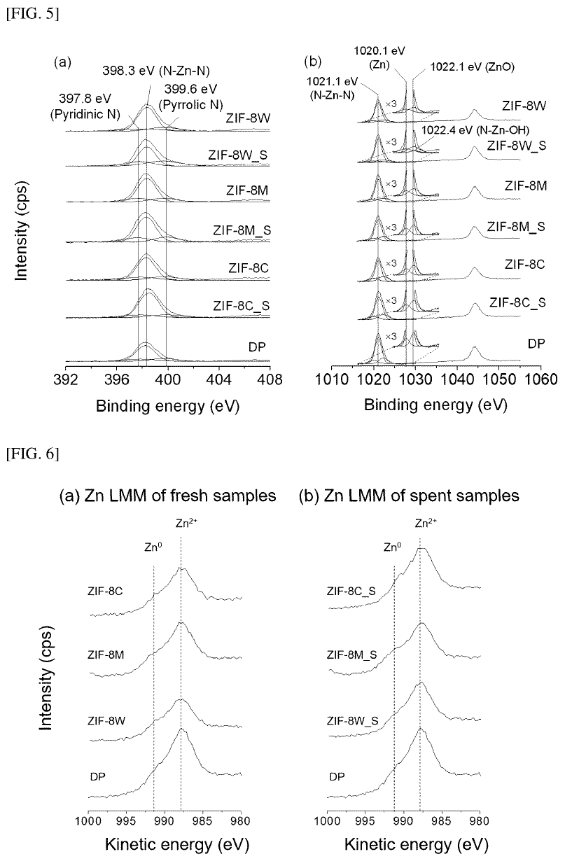

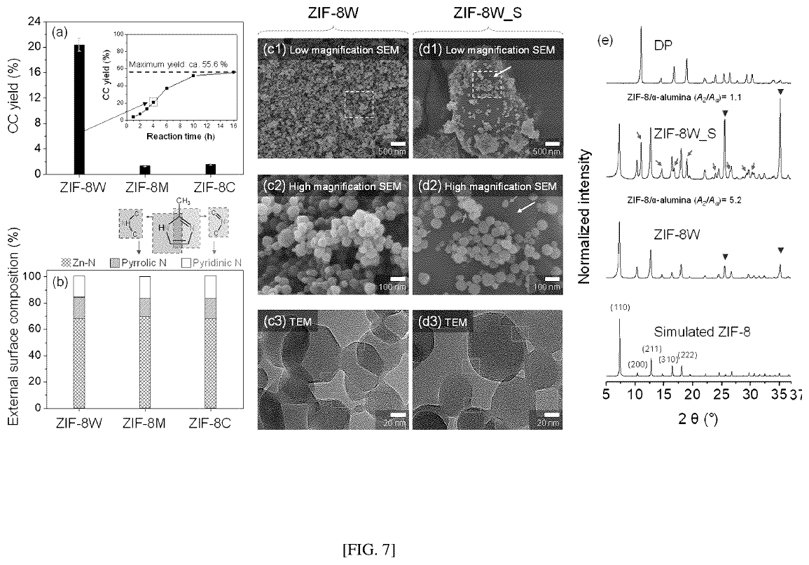

FIG. 4 shows XPS analysis results for ZIF-8W, ZIF-8W-S, and DP. For each compound in FIG. 4, FIGS. 4A1 to 4A3 represent XPS spectra of N 1s, FIGS. 4B1 to 4B3 represent XPS spectra of Zn 2p_{3/2}, and FIGS. 4C1 to 4C3 represent XPS spectra of O 1s. In this respect, FIGS. 4A1 to 4C1 represent ones for the ZIF-8W, FIGS. 4A2 to 4C2 represent ones for the ZIF-8W_S, and FIGS. 4A3 to 4C3 represent ones for the DP having deconvoluted curves, respectively. For comparison, the curve summing the deconvoluted curves is indicated as “SUM”. To show the appropriate fitting, the corresponding residual plots are shown below the experimentally obtained XPS spectra. For the residual plots, the normalized residual (R_{N}) was calculated by subtracting the experimentally measured XPS spectrum from the intensities summed from the deconvoluted curves and further dividing by the value of the experimentally measured XPS spectrum. FIG. 5 shows N is XPS spectra (FIG. 5A) and Zn 2p XPS spectra (FIG. 5B) of ZIF-8x and ZIF-8x_S (x=W, M, and C). For comparison, the results for DP have been added at the bottom. FIG. 6 shows XAES spectroscopy of Zn LMMs for fresh ZIF-8x (FIG. 6A) and spent ZIF-8x_S (x=W, M, and C) (FIG. 6B). For comparison in FIG. 6, the Auger electron spectra of the Zn LMM for DP are added at the bottom, and the peaks corresponding to Zn⁰ and Zn²⁺ are indicated by dotted lines.

FIGS. 4B1 to 4B3 and FIG. 5B show XPS results in three types of ZIF-8x (x=W, M, and C) and DP, and in FIG. 6, the presence of Zn metal was also supported by the corresponding XAES.

Further, Zn metal was found to be present in a very small amount by XPS and XAES analysis of intact ZIF-8 in previous studies. Although some metallic Zn species in the synthesis process may be formed with the help of the positive charges supplied by 2-methylimidazole, the presence of ZnO suggests facile oxidation. Next, considering that the ZnO single crystal (purity of 99.99%) exhibits some degree of Zn metal, it was assumed that the ZnO moiety, which may form on the surface due to inevitably exposed to ambient conditions, may reduce the Zn metal while preparing the XPS measurement. Nevertheless, the ratios of the metallic Zn species and ZnO in all four samples were almost the same regardless of fresh and spent forms. Therefore, it is conceivable that both of the metal Zn and ZnO do not contribute to the catalytic activity of the CO₂ cycloaddition reaction.

### 2. Properties of ZIF-8 Before and After Cycloaddition Reaction

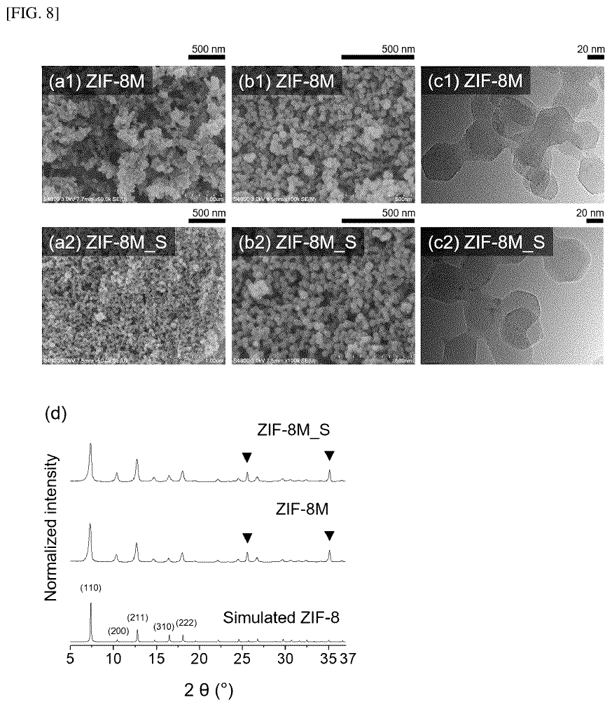

FIGS. 7 to 9 are views showing analysis of the properties of ZIF-8.

In FIG. 7, FIG. 7A shows CC yields of ZIF-8x (x=W, M, and C) when the carbon dioxide cycloaddition reaction is performed for 4 hours according to the CC yield of ZIF-8W at various reaction times (inset). FIG. 7B shows external surface compositions of nitrogen in ZIF-8x. FIGS. 7C1 and 7C2 show SEM images of ZIF-8W, FIG. 7C1 is a low magnification image, and FIG. 7C2 is a high magnification image. FIG. 7C3 is a TEM image of ZIF-8W. FIGS. 7D1 and 7D2 show SEM images of ZIF-8W_S, FIG. 7D1 is a low magnification image, and FIG. 7D2 is a high magnification image. FIG. 7D3 is a TEM image of ZIF-8W_S. FIG. 7E shows XRD patterns of ZIF-8W, ZIF-8W_S, and simulated ZIF-8 along with the dense phase (DP) particles, and for comparison, the highest peak in ZIF-8 or DP was used after normalizing the XRD patterns. The dashed line in the inset of FIG. 7A indicates the maximum yield (55.6%) under these reaction conditions. To facilitate understanding, a schematic diagram of 2-methylimidazole is added to the upper part of FIG. 7B. For clarity, descriptions of the images in FIGS. 7C1 to 7C3 and FIGS. 7D1 to 7D3 have been provided. Dashed rectangles in FIGS. 7C1 and 7D1 indicate the regions of high magnification SEM images in in FIGS. 7C2 and 7D2, respectively. Long-tailed arrows in FIGS. 7D1 and 7D2 indicate newly appearing particles in ZIF-8W_S.

Dashed rectangles in the TEM images of FIGS. 7C3 and 7D3 emphasize the morphological changes after the reaction. In FIG. 7E, tailed arrows indicate the newly formed XRD peaks, and inverted triangle marks indicate the XRD peaks of α-alumina used as an internal standard. In the case of ZIF-8W and ZIF-8W_S in FIG. 7E, the relative ratio (denoted by AZ/Aa) of ZIF-8 to α-alumina was obtained using the XRD peak regions of 7.3° and 35.1° corresponding to ZIF-8 and α-alumina, respectively.

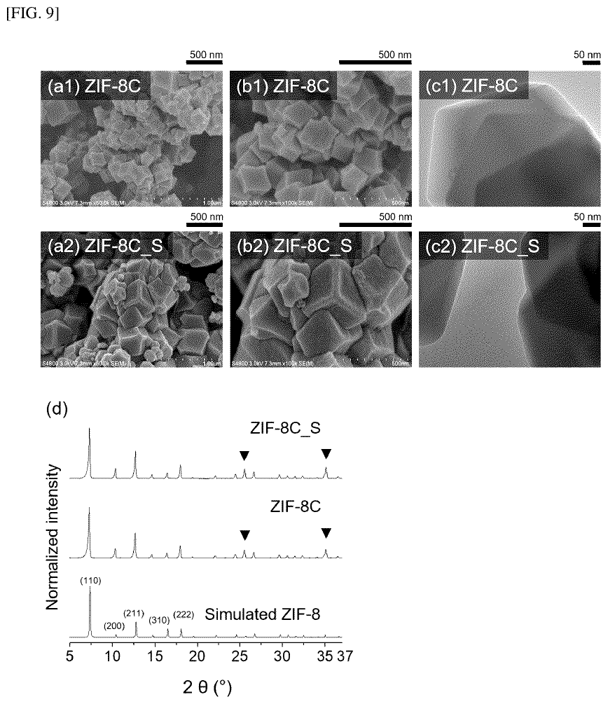

In FIG. 8, FIGS. 8A1 and 8B1 are SEM images for ZIF-8M, FIGS. 8A2 and 8B2 are SEM images for ZIF-8M_S, FIGS. 8A1 and 8A2 show low magnification images, and FIGS. 8B1 and 8B2 show high magnification images. FIG. 8C1 shows a TEM image for ZIF-8M, and FIG. 8C2 shows a TEM image for ZIF-8M_S. FIG. 8D shows XRD patterns of ZIF-8M and ZIF-8M_S along with the XRD pattern of simulated ZIF-8. Inverted triangle marks in FIG. 8D indicate α-alumina used as an internal standard.

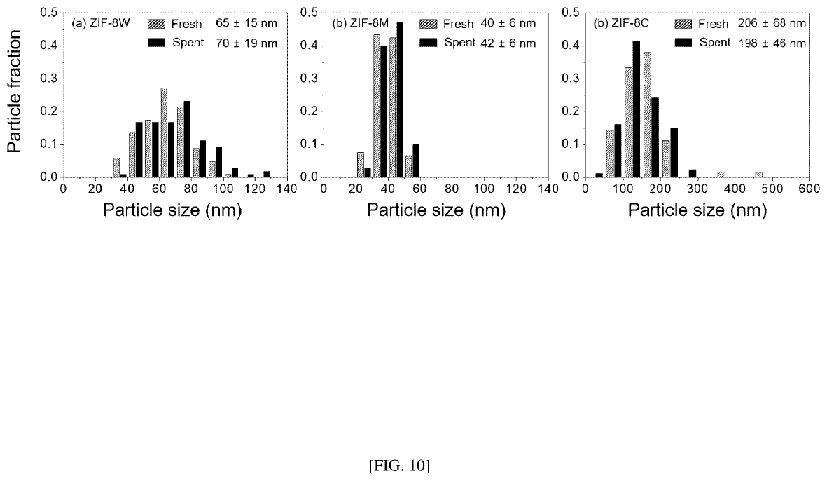

In FIG. 9, FIGS. 9A1 and 9B1 are SEM images for ZIF-8C, FIGS. 9A2 and 9B2 are SEM images for ZIF-8C_S, FIGS. 9A1 and 9A2 show low magnification images, and FIGS. 9B1 and 9B2 show high magnification images. FIG. 9C1 shows a TEM image for ZIF-8C, and FIG. 9C2 shows a TEM image for ZIF-8C_S. FIG. 9D shows XRD patterns of ZIF-8C and ZIF-8C_S along with the XRD pattern of simulated ZIF-8. Inverted triangle marks in FIG. 9D indicate α-alumina used as an internal standard.

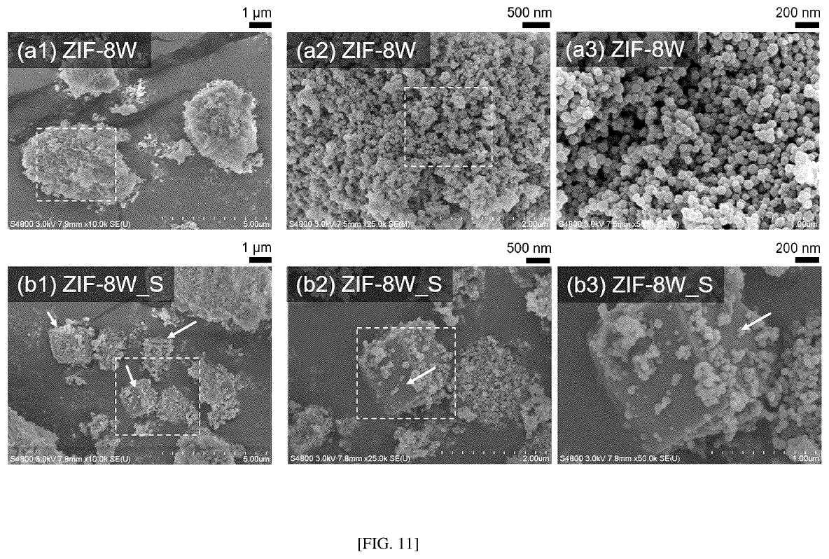

FIG. 10 shows size distributions of respective catalyst particles. The size distributions of ZIF-8W (FIG. 10A), ZIF-8M (FIG. 10B), and ZIF-8C (FIG. 10C) are shown for fresh (diagonally striped column) and spent (solid column) states for the respective catalyst particles. For size measurement, 100 particles were measured for each of the three types of ZIF-8 while considering the longest dimension. For consistency, particles that were too large (i.e., newly formed particles) in ZIF-8W_S were not measured.

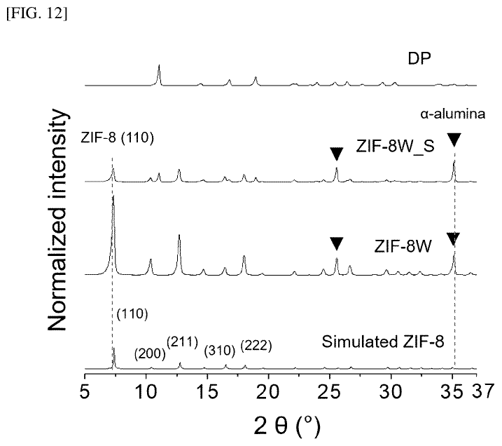

FIG. 11 shows SEM images for ZIF-8W and ZIF-8W_S. FIGS. 11A1 to 11A3 are ones for ZIF-8W, FIGS. 11B1 to 11B3 are ones for ZIF-8W_S, FIGS. 11A1 and 11B1 are low magnification images, and FIGS. 11A2 and 11B2 are high magnification images. The images of FIGS. 11A2 and 11B2 and FIGS. 11A3 and 11B3 are ones obtained by enlarging dashed rectangle portions in FIGS. 11A1 and 11A2 and FIGS. 11B1 and 11B2, respectively.

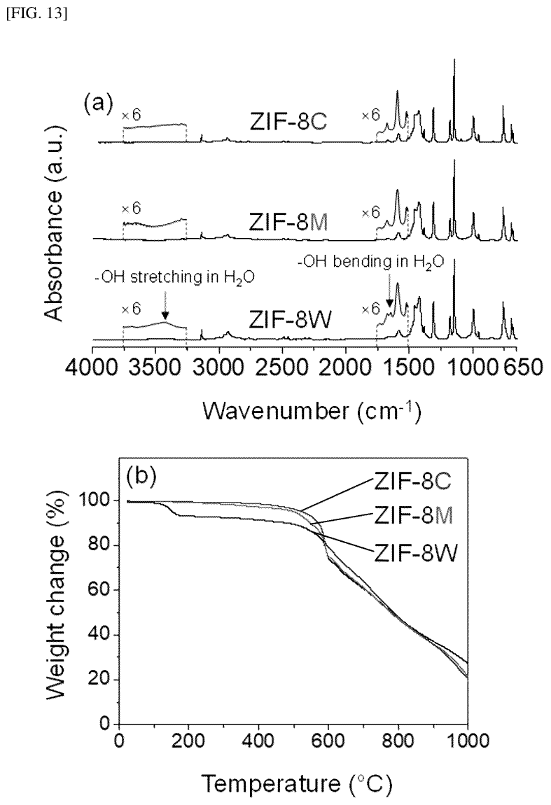

FIG. 12 is XRD patterns of ZIF-8W, ZIF-8W_S, and DP according to the simulated XRD pattern of ZIF-8. The XRD patterns for ZIF-8W and ZIF-8W_S in FIG. 12 are identical to those in FIG. 7E except for normalized intensities here. The normalized intensities here are ones in which the XRD peaks of ZIF-8-W and ZIF-8W_S are normalized to the α-alumina peak at 35.1°. The triangle marks indicate XRD peaks for α-alumina used as internal standard.

When fully integrated into the ZIF-8 framework, the Zn or N atoms were considered to be saturated and show no catalytic activity. However, in FIG. 7A showing the properties between the three types of ZIF-8x (x=W, M, and C), only ZIF-8W showed noticeable catalytic properties for cycloaddition reaction of ECH and CO₂ while showing a high CC yield of about 20.3% after 4 hours of reaction. Surprisingly, the other two types of ZIF-8 (ZIF-8M and ZIF-8C) showed CC yields of 1.2% and 1.5%, respectively, so that catalytic properties are shown slightly or are not shown. In addition, the CC yield of ZIF-8W increased monotonically with increasing reaction time, and reached a maximum yield of 55.4% after 16 hours of reaction time (inset of FIG. 7A). In conventional studies, the catalytic activity has been mainly contributed by coordinatively unsaturated Zn or unbonded N species presenting on the surface of the ZIF-8 structure. According to this, XPS analysis was performed in order to identify dissociated Zn or N species on the surface of ZIF-8x (x=W, M, and C) (FIG. 7B). In particular, the degrees of contribution of Zn and N species in the three types of fresh ZIF-8 catalysts were similar, and it is implied that the changes in physicochemical properties occurred during the CO₂ cycloaddition reaction.

Considering the similar properties and distributions of chemical species on the surface of the three types of ZIF-8, the different physicochemical properties of the catalysts were examined in order to find noticeable differences. The shapes/sizes and crystal structures of ZIF-8M (FIGS. 8A1 to 8C1, and 8D) were similar to those reported in previous studies, and commercially available ZIF-8C had the same physicochemical properties. (FIGS. 9A1 to 9C1, and 9D). Also, the shape/size of ZIF-8W, which showed noticeable catalytic performance (FIG. 7A), was similar to those (FIGS. 7C1 and 7C2) reported in conventional studies. In order to confirm the angular shape in appearance (FIG. 7C2) of ZIF-8W detected at SEM resolution, TEM analysis was additionally performed. The dashed rectangle in FIG. 7C3 clearly shows the angular shape of ZIF-8W. Finally, the particle sizes of ZIF-8x (x=W, M, and C) were measured to be 65±15 nm, 40±6 nm, and 206±68 nm, respectively (FIG. 10). In addition, XRD analysis also shows the pure ZIF-8 crystal structure of ZIF-8W (FIG. 7E). All three types of ZIF-8x (x=W. M, and C) are shown to have integrity with high crystallinity of ZIF-8 regardless of some different microscopic features (e.g., the more defined planes (FIG. 8C1) in ZIF-8M) shown from the TEM results. In this study, it was found that the cycloaddition reaction of CO₂ to ECH will occur mainly preferentially on the external surface of ZIF-8, and three types of ZIF-8x (x=W, M, and C) having the aforementioned particle sizes and external surface areas will become examples for understanding the unique catalytic activity of ZIF-8 based on some notable differences in their physicochemical properties. Considering that the size and crystallinity of ZIF-8W are larger than and similar to those of ZIF-8M, other physicochemical properties will be key to explaining the noticeable catalytic activity of ZIF-8W. The surface area for each catalyst is summarized in Table 3. In Table 3, the BET surface area (S_{B}ET) was calculated from N₂ physical adsorption isotherm data using the Brunauer-Emmett-Teller equation, the external surface area (Sₑₓₜₑᵣₙₐₗ) was calculated using the improved t-plot method, and the internal surface area (Sᵢₙₜₑᵣₙₐₗ) was calculated using Equation 3.

TABLE 3

| | S_{BET}^{a} (m² · g⁻¹) | | Sₑₓₜₑᵣₙₐₗ^{b}(m² · g⁻¹) | | Sᵢₙₜₑᵣₙₐₗ^{c}(m² · g⁻¹) | | Sₑₓₜₑᵣₙₐₗ/Sᵢₙₜₑᵣₙₐₗ | |
|---|---|---|---|---|---|---|---|---|
| Catalyst | Fresh | Spent | Fresh | Spent | Fresh | Spent | Fresh | Spent |
| ZIF-8W | 1390 ± 11 | 530 ± 1 | 184 | 106 | 1220 | 424 | 0.13 | 0.25 |
| ZIF-8M | 1592 ± 2 | 1501 ± 2 | 194 | 199 | 1398 | 1302 | 0.14 | 0.15 |
| ZIF-8C | 1626 ± 12 | 1473 ± 11 | 27 | 29 | 1599 | 1444 | 0.02 | 0.02 |

Sᵢₙₜₑᵣₙₐₗ=S_{BET}−Sₑₓₜₑᵣₙₐₗ [Equation 3]

Further, the properties of spent ZIF-8W (marked as ZIF-8W_S) were characterized in order to confirm whether the properties of ZIF-8W were changed during the CO₂ cycloaddition reaction (FIGS. 7D1 to 7D3, and 7E). For comparison, the properties of ZIF-8M_S and ZIF-8C_S were also examined (FIGS. 8A2 to 8C2, and 8D, and FIGS. 9A2 to 9C2, and 9D). ZIF-8W_S bears newly formed particles (indicated by arrows in FIGS. 7D1 and 7D2) and has a different shape compared to fresh ZIF-8W. SEM images of ZIF-8W and ZIF-8W_S at different magnifications in FIG. 11 clearly revealed ZIF-8W_S containing only newly formed particles (a size of 2 m), and this was much larger than that of original ZIF-8W particles. Also, XRD analysis shows that the newly formed particles have a different crystal structure (indicated by tailed arrows in FIG. 7E) and deviate from the ZIF-8 structure. In addition, it was recognized that the XRD peak intensities (indicated by inverted triangles in FIG. 7E) of α-alumina, which was used as an internal standard for rigorous analysis, increased compared to those of ZIF-8W. Therefore, considering that there is no change in the total weight of the particles after the reaction, the decrease in the XRD peak ratio of ZIF-8 (2θ=7.3°) to α-alumina (2θ=35.1°) proves that the ratio of the original ZIF-8W in ZIF-8W_S is reduced. For better demonstration, the XRD patterns of ZIF-8W and ZIF-8W_S were normalized by the α-alumina peak (FIG. 12), which clearly supported the reduced ratio of ZIF-8 after reaction, and thus supported the reduction degree of the original crystallinity. Thus, the new phase represents the one originated from fresh ZIF-8W after CO₂ cycloaddition reaction. Further, in addition to newly formed phases in ZIF-8W_S, SEM images indicate that there are some morphological changes in ZIF-8W after CO2 cycloaddition reaction in ZIF-8W_S (indicated by short-tailed arrows in FIG. 7D2). In addition, the SEM technique reveals the presence in the newly formed particles after CO₂ cycloaddition reaction (FIGS. 7C1 and 7D1, and FIG. 11). TEM analysis clearly shows the round surface of ZIF-8W_S (indicated by dashed rectangles in FIG. 7D3). Although there are differences between ZIF-8W and ZIF-8W_S, ZIF-8x and ZIF-8x_S (x=M and C) were similar to each other (FIGS. 8 and 9). In addition, the particle size of ZIF-8x_S (x=W, M, and C) was similar to those of the fresh materials except for ZIF-8W_S (i.e., the newly formed particles mentioned above) containing too large particles (FIG. 11).

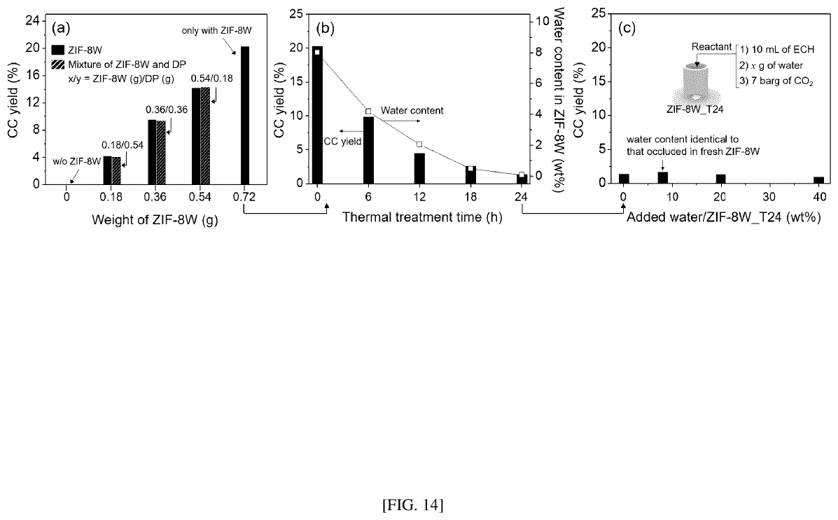

According to the conventional literature, the ZIF-8 structure can be transformed and reported in the presence of CO₂ and water vapor, and a similar transformation occurred in this study. Considering that water is not utilized for the CO₂ cycloaddition reaction, the transformation of ZIF-8W occurs due to its inherent properties. Especially compared to ZIF-8M and ZIF-8C, only ZIF-8W contained solvent (i.e., water) inside the pore structure. In order to confirm this, TGA and FT-IR analysis were performed on three types of ZIF-8 (FIG. 13). FT-IR spectroscopy revealed the presence of hydroxyl groups in ZIF-8W, thereby showing distinct features (FIG. 13A). In addition, TGA (FIG. 13B) shows that there is a loss of weight only in ZIF-8W while the temperature is being increased from 100° C. to 200° C. Not only reasoning based on the use of water in the synthesis of ZIF-8W, but also both properties indicate that only ZIF-8W contains water molecules within its pore structure. The occluded water molecules within ZIF-8W appear to be related to the transformation of the ZIF-8 structure, and the concomitant formation of active sites may explain the remarkable activity of ZIF-8W for CO₂ cycloaddition reaction.

### 3. Causes of ZIF-8W Activity for CO2 Cycloaddition

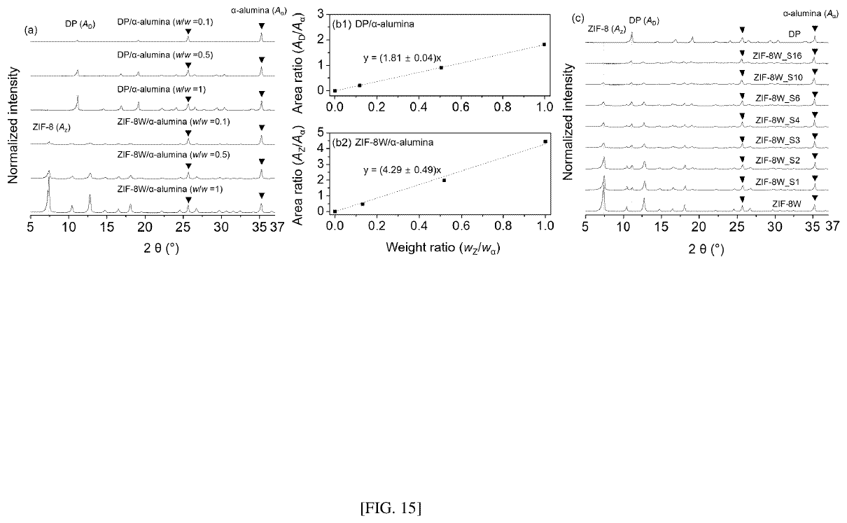

FIG. 14 shows results of experiments conducted by thermally treating ZIF-8W to remove water molecules or mixing with DP in order to confirm the catalytic properties of ZIF-8W. FIG. 14A shows CC yields in the cases of ZIF-8W (solid column) and mixtures of ZIF-8W and DP at different ratios (diagonally striped column). In this experiment, ZIF-8W were used in an amount of 0, 0.18, 0.36, 0.54, and 0.72 g, respectively, and ZIF-8W and DP were used as a mixing ratio of 0/0.72, 0.18/0.54, 0.36/0.36, 0.54/0.18, and 0.72/0 g, respectively. FIG. 14B shows the CC yield and water content of ZIF-8W thermally treated at 100° C. for various thermal treatment times. FIG. 14C shows CC yields of ZIF-8W thermally treated at 100° C. for 24 hours (ZIF-8W_T24) under reaction conditions in the presence of a certain amount of water (up to 40%). All reactions except for FIG. 14C in FIG. 14 were performed according to the reaction conditions below. 0 reactants: epichlorohydrin (ECH, 10 mL) and CO₂ (7 barg), 0 total weight of used catalysts: 0.72 g, and @reaction temperature and time: 70° C. and 4 hours. In FIG. 14C, water was added as a reactant to satisfy a predetermined ratio of water to ZIF-8W_T24 (0.72 g). In FIGS. 14B and 14C, water inside the catalyst was determined by TGA.

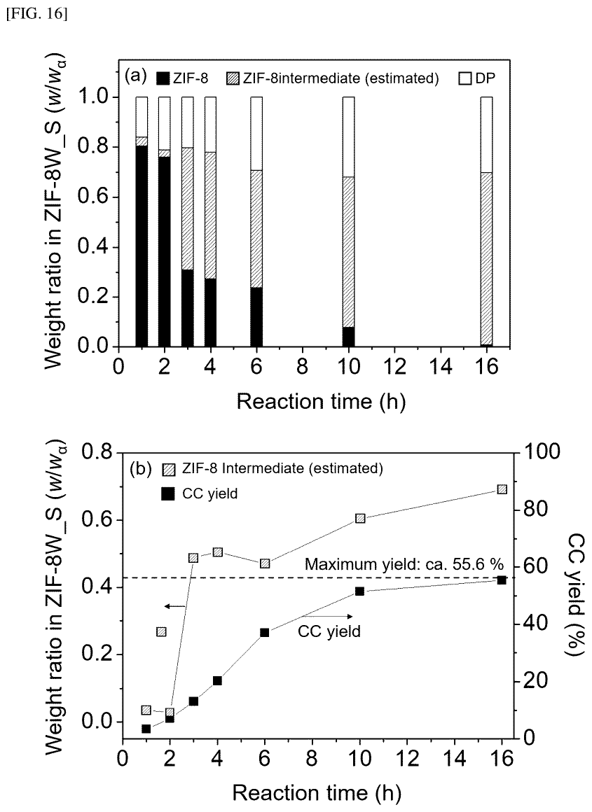

FIG. 15 shows XRD patterns measured by mixing ZIF-8W or DP with α-alumina at various ratios in order to find out the state of ZIF-8W having catalytic activity for carbon dioxide cycloaddition reaction. FIG. 15A shows XRD patterns of the mixtures of DP/α-alumina and ZIF-8W/α-alumina, which are mixed at different ratios (0.1, 0.5, and 1). FIG. 15B is a linear regression for the weight ratios of these mixtures, FIG. 15B1 is one for DP/α-alumina, and FIG. 15B2 is one for ZIF-8W/α-alumina. FIG. 15C shows XRD patterns of ZIF-8W recovered after performing the CO₂ cycloaddition reaction with different reaction times (1, 2, 3, 4, 6, 10, and 16 hours), respectively. For convenience, the spent catalyst is represented by ZIF-8W_Sx, where x represents the reaction time as 1, 2, 3, 4, 6, 10, or 16 hours. In FIGS. 15A and 15C, inverted triangle marks indicate α-alumina used as an internal standard. For comparison, FIG. 15C included XRD patterns for pure ZIF-8W and DP.

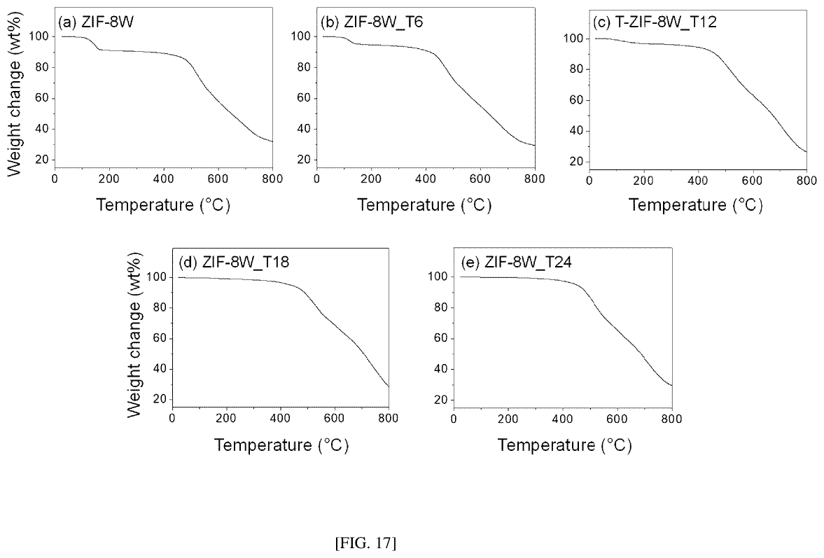

FIG. 16 is a graph in which the weight ratio of a ZIF-8 intermediate derivative is estimated from the weight ratio of ZIF-8 and DP in the catalyst after reaction, and shows the weight ratio and the CC yield of the ZIF-8 intermediate derivative according to the reaction time. FIG. 16A shows weight ratios (w/wα) for ZIF-8 (solid columns), ZIF-8W intermediate derivatives (estimates, diagonally striped columns), and DP (empty columns) in ZIF-8W_S recovered after performing reactions with different reaction times (1, 2, 3, 4, 6, 10, and 16 hours), respectively. FIG. 16B shows weight ratio and CC yield (shown in FIG. 7A) of the ZIF-8 intermediate derivative (estimate) in ZIF-8W S indicated as a function of reaction time.

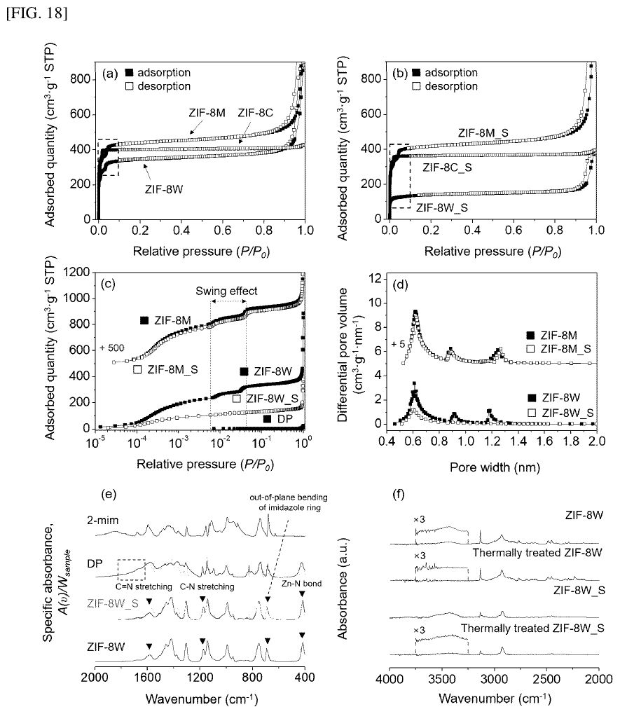

FIG. 17 shows TGA results of thermally treated ZIF-8W. They were indicated as ZIF-8W_Tx depending on the respective thermal treatment times, where Tx represents one subjected to hydrothermal treatment for x hours. Thermal treatment was performed at 100° C. for 0 hours (FIG. 17A), 6 hours (FIG. 17B), 12 hours (FIG. 17C), 18 hours (FIG. 17D), and 24 hours (FIG. 17E).

As an initial attempt to reveal the unique catalytic activity of ZIF-8W, the catalytic ability of newly formed particles from ZIF-8W (FIG. 14A) was examined. In order to simulate the particles shown in FIGS. 7D1 and 7D2, particles named for dense phase (DP), which can be explained by the previously mentioned newly appearing XRD peaks (FIG. 7E), were synthesized and used. In particular, this DP is similar to that reported in the conventional literature, and DP particles may be formed when ZIF-8 is exposed to an environment containing both CO₂ and gaseous water or when ZIF-8 is immersed in liquid water. For elucidation, the weight part of ZIF-8W and DP was systematically varied, but the total weight was fixed at 0.72 g, and the weight of ZIF-8x (x=W, M, and C) was used in performing the reaction shown in FIG. 7A. Obviously, the CC yield was almost the same as the result of reacting with the same amount of ZIF-8W. Therefore, the catalytic activity was related to the amount of ZIF-8W, not the amount of DP. Thus, DPs identical to fully transformed ZIF-8W did not appear to account for any catalytic activity for the CO₂ cycloaddition reaction. Therefore, the catalytic activity of ZIF-8W in CO₂ cycloaddition reaction is mostly related to intermediate derivatives. As inferred from the XRD patterns of ZIF-8W (FIG. 7E), the proportions of DP and ZIF-8 will increase and decrease after the reaction, respectively. In particular, the use of α-alumina as an internal standard is preferred for quantitative analysis. Referring to such approach, it was sought to measure the ratio of ZIF-8 intermediate derivative as a function for reaction time (FIG. 15). FIG. 16 shows that the ratios of the CC yield and ZIF-8 intermediate derivative follow a tendency similar to a function of reaction time. This indicates that the possible active component of the ZIF-8 intermediate derivative in this study originates from the CO₂ cycloaddition reaction.

As a next step for revealing the cause of the catalytic activity of ZIF-8W, the transformation of ZIF-8W was examined during the CO₂ cycloaddition reaction. Considering that ZIF-8W may induce the formation of DP particles during the CO₂ cycloaddition reaction, thermal treatment was performed at about 100° C. for different times to reduce the amount of water molecules occluded inside ZIF-8W. The final amount of water was accurately determined by considering the weight change between 100° C. and 170° C. in the TGA profile (FIG. 17). For convenience, the thermally treated ZIF-8 is denoted as ZIF-8W_Ty, where y represents the thermal treatment time (h). The catalytic activity corresponding to each thermally treated ZIF-8W shows that to the CC yield decreased monotonically with the increase in the duration of thermal treatment (i.e., decreased water content) (FIG. 14B). In addition, the same amount of water as the amount of water inside the synthesized ZIF-8W was added to the completely dried ZIF-8 (i.e., ZIF-8W_T24) and supplied to the reactants. Surprisingly, the CC yield was not related to the content of added water, whereas a catalytic activity similar to the yield of ZIF-8W_T24 was shown (FIG. 14C). Even with the addition of up to 40 wt % of water (vs. about 8 wt % of water inside as-synthesized ZIF-8W), the corresponding catalytic activity remained as low as that of ZIF-8W_T24. Considering that it is very difficult to put water molecules into the pores of ZIF-8 apparently at a pressure of 19 MPa or less due to its hydrophobic properties, water molecules outside the pore structure of ZIF-8 do not contribute to forming active sites for the desired catalytic activity. More information on the negligible water adsorption on fully dried ZIF-8x (x=W, M, and C) is shown as graphs in FIG. 2 and described above. This strongly indicates that the water molecules occluded in the ZIF-8 pore structure are the key to form the active site of the catalytic CO₂ cycloaddition reaction using a catalyst. In addition, the fact that the CC yield monotonically decreases with the decrease of water molecules (FIG. 14B) indicates that the generation of active sites depends on the water content. Thus, the other two types of ZIF-8, which do not contain water molecules, show no appreciable catalytic activity for the CO₂ cycloaddition reaction.

### 4. Active Site of ZIF-8W Acting as Catalyst in CO₂ Cycloaddition Reaction

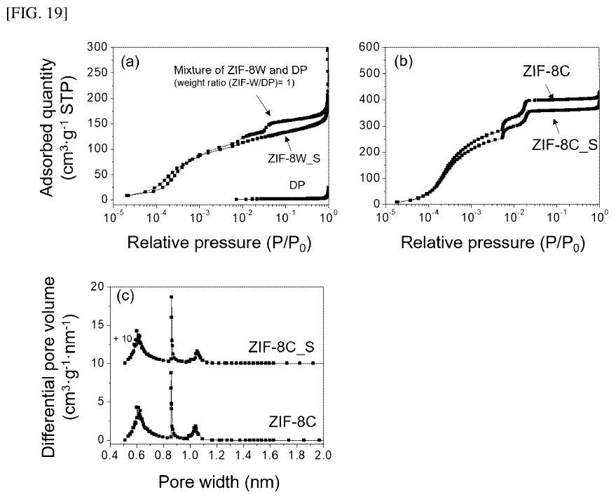

FIG. 18 shows results of measuring N₂ adsorption desorption isotherms, pore distributions, and FT-IR of respective samples in order to find out the site acting as a catalyst in ZIF-8W. Here, FIG. 18A shows N₂ adsorption-desorption isotherms of ZIF-8x, and FIG. 18B shows N₂ adsorption-desorption isotherms of ZIF-8x_S (x=W, M, and C). FIG. 18C shows N₂ adsorption-desorption isotherms of ZIF-8x and ZIF-8x_S (x=W and M) on a logarithmic x-axis, where the result for DP is added for comparison. FIG. 18D shows pore size distribution of ZIF-8x and ZIF-8x_S (x=W and M). FIG. 18E shows FT-IR spectra of ZIF-8W, ZIF-8W_S, DP, and 2-methylimidazole (2-mim). For C═N stretching, C—N stretching, bending of imidazole ring, and Zn—N bond, respectively, inverted triangles were marked and tagged on the corresponding peaks. In FIG. 18E, the samples were prepared in the form of pellets by pressing a mixture of the sample and KBr. For fair comparison, all samples in FIG. 18E were normalized by sample weight, and FT-IR for ZIF-8W/ZIF-8W_S and ZIF-8W_S/DP were shown together. For helping understanding, a tailed arrow in FIG. 18E is included in order to indicate some properties of the DP (looks like a broad hump). FIG. 18F shows FT-IR spectra of ZIF-8W, ZIF-8W_S, and thermally treated samples thereof. In FIG. 18F, one side of the samples was deposited with pure KBr pellets.

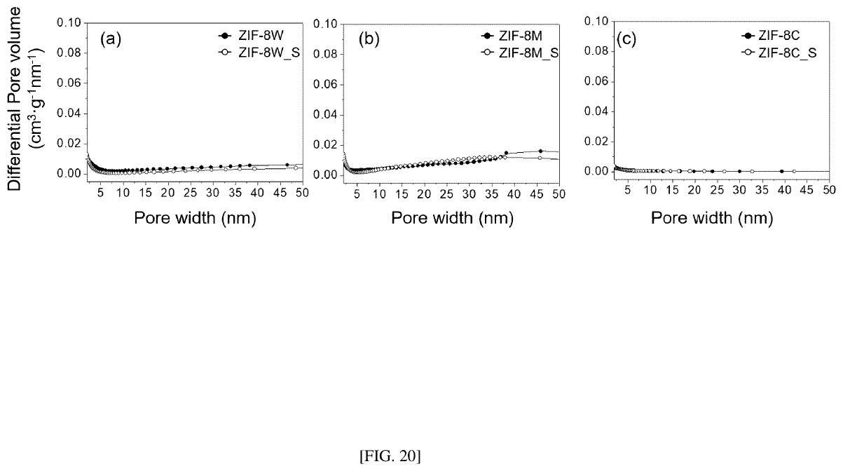

FIG. 19A shows N₂ adsorption-desorption isotherms for ZIF-8W_S, DP, and a mixture of ZIF-8W and DP on a logarithmic x-axis. FIG. 19B shows N₂ adsorption-desorption isotherms of ZIF-8C and ZIF-8C_S on a logarithmic x-axis. FIG. 19C shows micropore size distribution in ZIF-8C and ZIF-8C_S ranging from 0.4 nm to 2.0 nm.

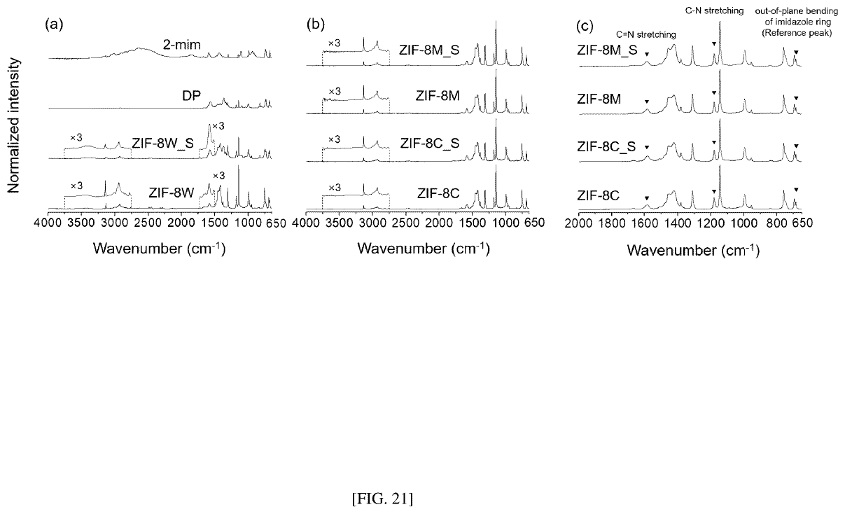

FIG. 20 shows mesopore size distributions of ZIF-8W (FIG. 20A), ZIF-8M (FIG. 20B), ZIF-8C (FIG. 20C), and respective spent catalysts thereof (ZIF-8x_S; x=W, M, and C) in the range of 2 nm to 50 nm. The samples were each marked with a tag.

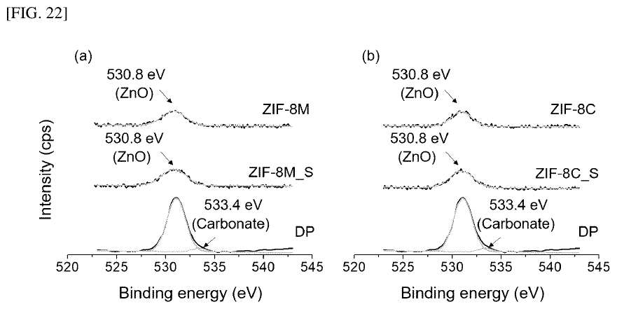

FIG. 21 shows FT-IR spectra of the respective samples. FIG. 21A shows FT-IR spectra of ZIF-8W, ZIF-8W_S, DP, and 2-methylimidazoe (2-mim). FIG. 21B shows FT-IR spectra of ZIF-8M, ZIF-8C, and spent catalyst states thereof. FIG. 21C shows FT-IR spectra of ZIF-8M, ZIF-8C, and spent catalysts thereof in the range of 2,000 to 650 cm⁻¹. For each of C═N stretching, C—N stretching and bending of the imidazole ring, the corresponding peak is marked with an inverted triangle and a tag. For each of C═N stretching, C—N stretching, and bending of imidazole ring, inverted triangles and tags were marked on the corresponding peaks. All FT-IR spectra in FIGS. 21A to 21C were normalized to the peak at 684 cm⁻¹ corresponding to bending of the imidazole ring. For comparison in FIG. 21C, FT-IR spectra of ZIF-8M and ZIF-8C are overlapped on peaks corresponding to those of catalysts in spent states thereof.

Although the effect of occluded water molecules on the ZIF-8 pore structure has been revealed, the chemical origin of the catalytic activity has not still been revealed. Since the CO2 cycloaddition reaction may be carried out in the presence of Lewis acids, bases or both, nitrogen and zinc in the Zn—N bond have little activity in this reaction. It was assumed that the active site might be related to newly formed N and/or Zn species that originate from Zn—N binding and apparently accompany structural transformation of ZIF-8. To this end, N₂ adsorption-desorption isotherms of ZIF-8x and ZIF-8x_S (x=W, M, and C) were examined in order to show structural differences (FIGS. 18A and 18B). ZIF-8x showed typical type 1 adsorption behavior, and a unique stepwise change appeared at low P/P₀ values (between 6.0×10⁻³ and 4.0×10⁻²) regardless of the synthesis route (indicated as a dashed rectangle in FIG. 18A). ZIF-8 exhibits structural flexibility through imidazolate (IM) that is linked to zinc when gas molecules are adsorbed, and this is a swing effect, which is a phenomenon that is reflected by a low P/P₀ value and a noticeable increase in adsorption of N₂ at 77 K. Such a stepwise change in N₂ adsorption may be used to confirm the presence of Zn-IM linkages in ZIF-8. Although the hysteresis loops in ZIF-8W and ZIF-8M are closed at different relative pressures, they seem to apparently originate from extrinsic particle agglomeration and thus will not affect any physicochemical or catalytic properties. In addition, noted was that there was a slight discrepancy for the BET surface area (see Table 2) for ZIF-8W in the N₂ adsorption isotherm (obtained after degassing at 80° C.). However, it was confirmed that the micropores were similar between ZIF-8W and ZIF-8M at 200° C. which is a higher degassing temperature (FIG. 3 and Table 2). Together with the aforementioned XRD results, N₂ physical adsorption shows high crystallinity comparable to all three types of ZIF-8 used in this study (Tables 2 and 3).

In addition, the general adsorption isotherm trend was maintained in the spent catalysts (i.e., ZIF-8x_S, x=M and C). However, ZIF-8W_S showed significantly reduced adsorption. Therefore, the BET surface area of ZIF-8W significantly decreased from 1,390 m²·g⁻¹ to 530 m²·g⁻¹ after reaction compared to those of the other two types of ZIF-8. For reference, ZIF-8M decreased from 1,592 m²·g⁻¹ to 1,501 m²·g⁻¹, and ZIF-8C decreased from 1,626 m²·g⁻¹ to 1,473 m²·g⁻¹. In addition, the above-mentioned unique stepwise change was not seen in ZIF-8W_S (indicated by the dashed rectangle in FIG. 18B). To facilitate the comparison in FIG. 18C, N₂ adsorption isotherms of ZIF-8W and ZIF-8W_S are shown together with those for ZIF-8M, ZIF-8M_S, and DP. The plots in the exponential range clearly showed a two-step change in P/P0 of about 6.0×10⁻³ and 4.0×10⁻² for ZIF-8W, ZIF-8M, and ZIF-8M_S. In contrast, ZIF-8W_S showed not only a decrease in adsorption, but also the absence of stepwise adsorption (FIG. 18C). Considering the negligible DP's N₂ adsorption capacity arising from the complete structural transformation of ZIF-8W during the CO₂ cycloaddition reaction, the significant reduction of N₂ adsorption in ZIF-8W_S is because of the collapse of the original structure inducing micropore reduction during formation of DP (FIG. 7E). Conversely, ZIF-8x (x=M and C), whose structures are maintained even after the reaction without formation of DP (FIGS. 8D and 9D), may maintain the original N₂ adsorption capacity (FIGS. 18A and 18B). Nevertheless, considering that the physical mixture of ZIF-8W and DP possesses stepwise adsorption behavior (FIG. 19A), the presence of DP alone cannot explain the absence of stepwise adsorption behavior in ZIF-8W_S. To examine the presence and absence of stepwise adsorption behavior before and after the reaction, the micropore size distribution was considered (FIG. 18D). ZIF-8W, ZIF-8M, and ZIF-8M_S have three peaks at 0.6, 0.9, and 1.2 nm. Among them, the first peak at 0.6 nm is related to N₂ adsorption in the micropore structure of ZIF-8, and the two peaks at 0.9 and 1.2 nm are related to additional N₂ adsorption by the swing effect. However, as expected, the two peaks at 0.9 and 1.2 nm did not exist in ZIF-8W_S. In addition, the adsorption behaviors of ZIF-8C and ZIF-8C_S (FIGS. 19B and 19C) were similar to those of ZIF-8M and ZIF-8M_S. Only ZIF-8W was transformed to the new dense phase, and the resulting ZIF-8W did not show stepwise adsorption after CO₂ cycloaddition reaction. From these results, it was shown that the structural transformation of ZIF-8W to the intermediate phase prior to the DP phase is related to the creation of an active site through dissociation of the Zn-IM linkage. In addition, the fact that no additional micropores and mesopores are formed in any of the three types of ZIF-8 after the reaction (FIG. 18D, FIG. 19C, and FIG. 20) means that structural decomposition started together with the dissociation of the Zn—N bond inside the ZIF-8 structure.

FT-IR analysis of ZIF-8W, ZIF-8W_S and DP was referred to in order to examine the discrepancies in chemical properties in addition to physical pore properties (FIG. 18E and FIG. 21A). A FT-IR spectrum of 2-methylimidazole (2-mim) is also shown in FIG. 18E. For comparison, all FT-IR spectra shown in FIG. 18E were normalized to the weight of the sample. In addition, since the imidazole ring is independent of the dissociation of the Zn—N bond, the FT-IRs collected in the ATR mode and shown in FIG. 21 were normalized to the peak intensities of the bending vibration of the out-of-plane bending of the imidazole ring indicated by the inverted triangles (refer to the tag in the drawing, 684 cm⁻¹) shown in the reference peaks. It is noticed from this that after the CO₂ cycloaddition reaction, the intensity of the C═N stretching peak (indicated by an inverted triangle at 1,578 cm⁻¹ in FIG. 18E) increased, whereas the intensities of the C—N stretching peak (indicated by an inverted triangle at 1,180 cm⁻¹ in FIG. 18E) and the Zn—N bonding peak (indicated by an inverted triangle at 420 cm⁻¹ in FIG. 18E) decreased. In contrast to ZIF-8W, the FT-IR spectra of ZIF-8M and ZIF-8C did not change after the CO₂ cycloaddition reaction (FIGS. 21B and 21C). Although the possibility of increased peaks of C═N stretching arising from DP particles in ZIF-8W_S cannot be completely ruled out, the overlapped FT-IR spectra of ZIF-8W_S and DP in FIG. 18E suggest that the increased C═N stretching peak mainly originated from the creation of additional C═N bonds at the expense of dissociation of Zn—N bonds in ZIF-8W. Otherwise, a prominent broad hump corresponding to DP should have been observed in ZIF-8W_S. Thus, FT-IR analysis indicates that the N species in the Zn-IM linkage became pyridinic containing C═N bonds during the reaction time. Although the decrease of the FT-IR Zn—N bond peak was not significantly reduced for ZIF-8W_S (FIG. 18E), it can be explained that the Zn—N bond was broken from the marked increase in the peak due to the C═N bond. In addition, this trend was consistent with both XRD and N₂ physical adsorption results, which also appeared in damaged ZIF-8 structure in ZIF-8W_S.

Considering that the broad peak was shown additionally at about 3,400 cm⁻¹ for ZIF-8W_S in FIG. 21 after the reaction due to N—H and O—H groups, pyrrolic N species were also formed. In particular, the pyrrolic N species were mostly formed after dissociation of the Zn—N bond and subsequent protonation, suggesting that they are related to some proton species. Considering that the transformation of ZIF-8W into the dense phase results from the dissociation of Zn—N bonds in the presence of water, the additional protons that generated pyrrolic N species are closely related to the occluded water molecules. Protons in the pyrrolic N species seem to originate from water molecules occluded inside ZIF-8W. Therefore, it is expected that other hydroxyl groups apparently generating from water molecules are found in the FT-IR spectrum of ZIF-8W_S. Broad FT-IR spectra ranging from 4,000 to 2,000 cm⁻¹ revealed the presence of additional hydroxyl groups in ZIF-8W_S (FIG. 18F), but these were present along with peaks corresponding to N—H bonds. For rigorous analysis, the FT-IR spectra of thermally treated ZIF-8W and ZIF-8W_S were compared in FIG. 18F. In particular, the thermally treated ZIF-8W_S still contained hydroxyl groups that could not be removed by thermal treatment. Through a comprehensive property analysis of fresh and spent ZIF-8x (x=W, M, and C), it was concluded that the only structural transformation of ZIF-8W during CO₂ cycloaddition reaction arises from the dissociation of Zn—N bonds by water molecules occluded within the ZIF-8W pore structure. In addition, the dissociation of the Zn—N bond induces the simultaneous formation of Zn or N species, and this results in activation of the catalytic activity of ZIF-8W in the process of structural transformation to DP.

### 5. Chemical Species on the External Surface of ZIF-8W

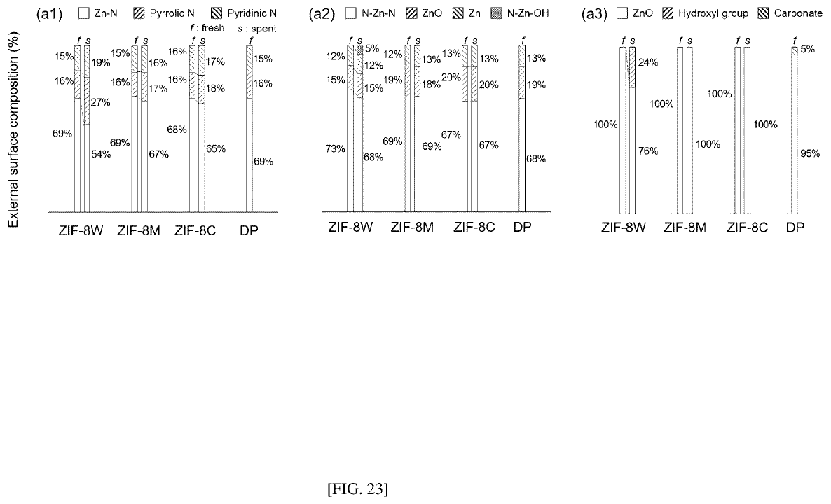

FIG. 22 shows XPS results for O 1s of ZIF-8M, ZIF-8M_S, and DP (FIG. 22A), and ZIF-8C, ZIF-8C_S, and DP (FIG. 22B).

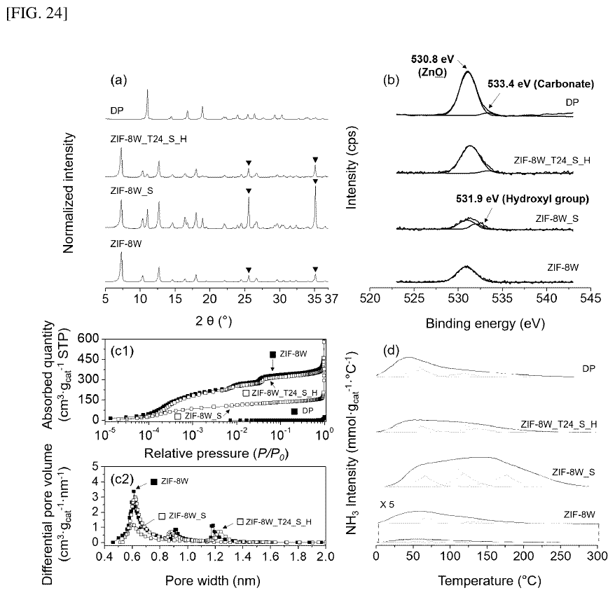

FIG. 23 shows the compositions of elements on the surface of the respective samples.

The external surface compositions are shown for nitrogen (FIG. 23A1), zinc (FIG. 23A2), and oxygen (FIG. 23A3) in ZIF-8x and ZIF-8x_S (x=W, M, and C). In FIGS. 23A1 to 23A3, the results for DP were added for comparison. The external surface compositions for nitrogen, zinc, and oxygen in ZIF-8x, ZIF-8x-S, and DP were obtained by deconvoluting the XPS spectra for N 1s, Zn 2p_{3/2}, and O 1s, respectively.

Acid-base sites may be present on the external surface of ZIF-8 or at their defective sites. Catalytic reactions such as CO₂ cycloaddition reaction and transesterification to ZIF-8 or ZIF-67 are promoted at active sites on the external surface of the catalysts (not their micropores). Although the noticeable catalytic activity of ZIF-8W is attributable to newly generating Zn and N species through the dissociation of Zn—N bonds, the actual active component responsible for the catalysis of the CO₂ cycloaddition reaction remains unclear. As mentioned above, the CO₂ cycloaddition reaction to ECH will occur mainly preferentially on the external surface of ZIF-8, and the study of the chemical species on the external surface of ZIF-8 by XPS analysis is required. In particular, the resulting properties should be understood to be consistent and complementary with the physicochemical properties of the various characterizations described above. First, in order to confirm the active site of ZIF-8W for CO₂ cycloaddition reaction, XPS analysis of ZIF-8W and ZIF-8W_S was used to refer to chemical species on the external surface (FIG. 4). In particular, it was concentrated on to track the XPS spectra of N 1s, O 1s, and Zn 2p. For comparison, XPS analysis of DP and two other types of fresh and spent ZIF-8 (ZIF-8x and ZIF-8x_S; x=M and C) was performed (FIGS. 4, 5 and 22). In particular, the good deconvolution of the XPS spectra of ZIF-8W, ZIF-8W_S, and DP supported by almost near-zero residual values shows the quantitative proportions of chemical species associated with Zn, N, and O atoms. Fitting parameters and standard deviations that correspond thereto are summarized in Table 4 below. The chemical compositions of the external surfaces of ZIF-8x, ZIF-8x_S (x=W, M, and C), and DP are summarized in FIGS. 23A1 to 23A3.

TABLE 4

| | | Samples | | |
|---|---|---|---|---|
| Chemical species | | ZIF-8W | ZIF-8W_S | DP |
| Zn | Metal Zn at 1,020.1 eV | 6005 ± 486.2 | 6601 ± 374.2 | 7372 ± 1029 |
| species | N—Zn—N at 1,021.1 eV | 31644 ± 466.33 | 36884 ± 544.36 | 38742 ± 1053.1 |
| (area) | ZnO at 1,022.1 eV | 7312 ± 547.2 | 8742 ± 457.2 | 10802 ± 831.8 |
| | N—Zn—OH at 1,022.4 eV | N/A | 2694 ± 597.8 | N/A |
| N | Pyridine-based N at | 3566 ± 447.4 | 5027 ± 423.4 | 2134 ± 423.3 |
| species | 397.8 eV | | | |
| (area) | Zn—N at 398.3 eV | 18324 ± 614.67 | 14193 ± 492.48 | 14232 ± 482.7 |
| | Pyrrole-based N at | 3683 ± 289.7 | 6660 ± 327.6 | 2113 ± 158.1 |
| | 399.6 eV | | | |
| O | ZnO at 530.8 eV | 9359 ± 171.9 | 5786 ± 325.1 | 24513 ± 173.6 |
| species | Hydroxide at 531.9 eV | N/A | 1839 ± 318.6 | N/A |
| (area) | Carbonate at 533.4 eV | N/A | N/A | 1174 ± 161.7 |

The peak at 398.3 eV accounts for the saturated N species participating in the Zn—N bonding of ZIF-8W. Meanwhile, the peaks at 399.6 eV and 397.8 eV correspond to the pyrrolic (because of N—H) and pyridinic (because of C═N) N species, respectively, which seem to be formed by the dissociation of the Zn—N bond (FIG. 4A1). In addition, the peak at 1,021.1 eV corresponds to the saturated Zn species connected with the N atom of ZIF-8W, whereas other Zn species such as metallic zinc (at 1,020.1 eV) and zinc oxide (at 1,022.1 eV) are also present (FIG. 4B1). The presence of zinc oxide is also supported by the 0 is XPS spectrum (FIG. 4C1). The XPS results of ZIF-8W are similar to those of ZIF-8M and ZIF-8C (FIGS. 5 and 22). The presence of metallic zinc may be confirmed by X-ray-excited Auger electron spectroscopy (XAES) for all samples (FIG. 6). In addition, although not discussed, the minor presence of some metallic Zn species in ZIF-8 was revealed by XPS and XAES in conventional studies. In contrast to the fresh samples, the XPS results of the spent catalysts (ZIF-8x_S; x=W, M, and C) show distinct features. In particular, the change (i.e., between ZIF-8W and ZIF-8W_S) in the XPS results of ZIF-8W during the CO₂ cycloaddition reaction was clear, but the other two types of ZIF-8 showed similar XPS results in both of fresh and spent ones (FIGS. 23A1 to 23A3). The three types of N species in ZIF-8W_S were the same as those in ZIF-8W, but the respective proportions were changed after the CO₂ cycloaddition reaction (FIGS. 4A1 and 4A2, and FIG. 23A1).

In the delicate peak deconvolution revealed for ZIF-8W in FIG. 23A1, the ratios of pyrrolic N species and pyridinic N species increased after the reaction, whereas the ratio of saturated N species decreased. Such a trend was consistent even with the FT-IR spectra of ZIF-8W and ZIF-8W_S (FIGS. 18E and 21A). These complementary characterizations indicate that a significant dissociation of Zn—N bonds occurs only in ZIF-8W, which is associated with a change in the ratio of N species after CO₂ cycloaddition reaction. Therefore, the distribution of Zn species in ZIF-8W and ZIF-8W_S was also different. These contained three types of Zn species (metallic zinc, nitrogen-linked zinc, and zinc oxide), but ZIF-8W_S has another small amount of Zn species at a higher binding energy (1,022.4 eV) (FIG. 4B2 and FIG. 23A2). The deconvolution of the main peak in FIG. 4B2 shows the ratio of Zn species corresponding to N—Zn—N reduced after the reaction, but that of zinc oxide and metallic zinc remained almost constant (FIG. 23A2). Therefore, it is reasonable to think that the newly formed Zn species corresponding to the peak at 1,022.4 eV originate from the dissociation of the Zn—N bond of ZIF-8W during the reaction. In this part, it was speculated that the coexistence of metallic Zn and ZnO species on the external surface could be attributed to the decrease in the proportion of ZnO by vacuuming for the XPS measurement. This speculation originates from the presence of small amounts of metallic Zn species in the XPS and XAES spectra of ZnO single crystals (99.99%).

FT-IR analysis revealed that there are some hydroxyl groups in ZIF-8W_S (FIG. 18F). Besides N and Zn species, the presence of O atoms in XPS analysis was further examined. In ZIF-8W_S, O species were present in the state of zinc oxide (at 530.8 eV) and hydroxyl group (at 531.9 eV). The presence of hydroxyl groups in the O 1s XPS spectra was consistent with Zn species linked to hydroxyl groups (FIGS. 4B1 and 4B2, FIGS. 4C1 and 4C2, and FIGS. 23A2 and 23A3), as supported by an additional peak at 1,022.4 eV in the Zn 2p XPS spectrum of the aforementioned ZIF-8W_S. In XPS analysis, the binding energies of chemical species in the same oxidation state are related to the electronegativity of the other binding atoms. Therefore, Zn species bound to atoms of higher electronegativity have higher binding energies (i.e., binding energies of N—Zn—N, ZnO, and Zn(OH)₂ are 1,021.1 eV, 1,022.1 eV, and 1,022.7 eV respectively), and the newly formed Zn species exhibiting an XPS peak of up to 1,022.4 eV may correspond to N—Zn—OH.

In the FT-IR and XPS analyzes for ZIF-8W and ZIF-8W_S, a phenomenon that may occur in ZIF-8W during the CO₂ cycloaddition reaction (FIG. 1) is proposed. The Zn—N bonds in ZIF-8W are likely dissociated by water molecules occluded inside the ZIF-8W structure. This results in the formation of dissociated N and Zn species. Dissociated N species may be pyrrolic and pyridinic, whereas dissociated Zn species may exist in the N—Zn—OH form. Since no additional micropores/mesopores are formed after the reaction, such species will mainly be present on the external surface (FIG. 18D, FIG. 19C, and FIG. 20). In particular, the N—Zn—OH species are consistent with species that may be present due to hydrolysis of ZIF-8. Consequently, the unique catalytic activity of ZIF-8W appears to originate from dissociated N or Zn species. In this part, it was assumed that the water molecules occluded inside ZIF-8W have an unstable state.

### 6. The Role of Water Molecules for the Formation of Active Sites in ZIF-8W

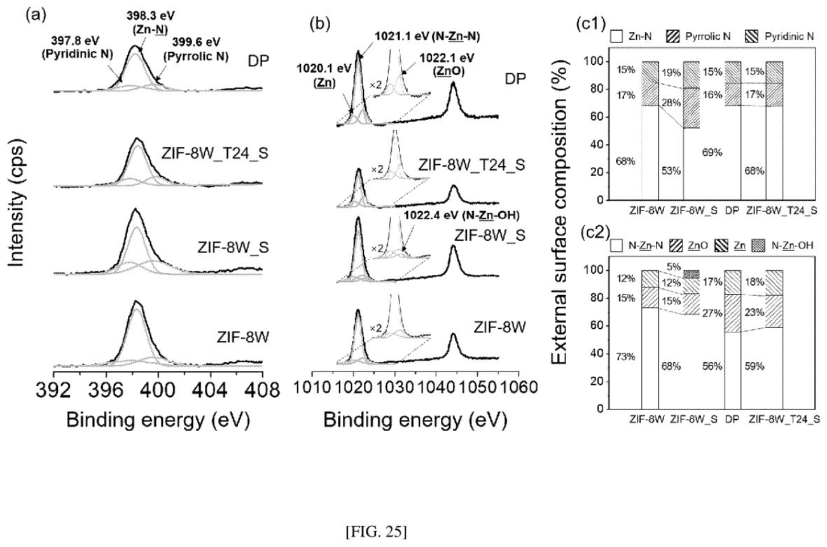

FIG. 24 is to find out how ZIF-8W and water molecules form an active site. For spent ZIF-8W (ZIF-8W_S) and spent ZIF-8W_T24, respectively, XRD patterns (FIG. 24A), O 1s XPS spectra (FIG. 24B), N₂ adsorption-desorption isotherms (FIG. 24C1), and micropore size distributions in the size range of 0.4 to 2.0 nm (FIG. 24C2) are shown. Here, for ZIF-8W_T24, the same amount of water molecules as the water molecules (8 wt %) occluded inside ZIF-8W was additionally added to the reaction (indicated as ZIF-8W_T24_S_H). For comparison, the analysis results of ZIF-8W and DP are added to FIGS. 24A to 24D. The inverted triangle marks in FIG. 24A represent α-alumina as an internal standard.

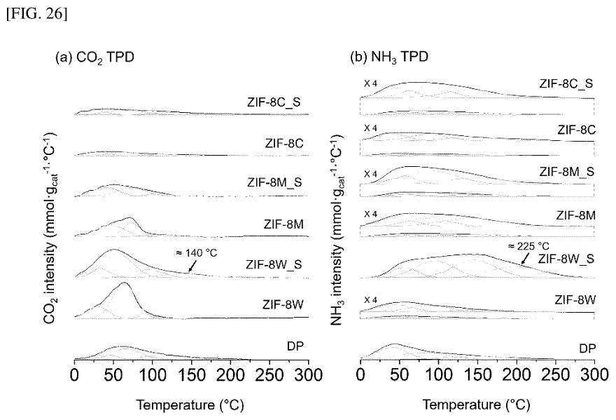

FIG. 25 shows XPS spectra and external surface compositions for N is (FIG. 25A) and Zn 2p (FIG. 25B) in ZIF-8W, ZIF-8W_S, ZIF-8W_T24_S_H, and DP.

Completely dried ZIF-8W_T24 did not show any noticeable catalytic activity in CO₂ cycloaddition reaction in which water (water fed up to 40 wt %) was present (FIG. 14C). To understand the role of occluded water in the unique catalytic activity of ZIF-8W, physicochemical properties of ZIF-8W (containing 8 wt % of water inside) and ZIF-8W_T24 (containing 8 wt % of water outside) after CO₂ cycloaddition reaction were examined. For convenience, the spent ZIF-8W_T24 under reaction conditions containing water (8 wt % of water) is indicated as ZIF-8W_T24_S_H, where the character H is added to indicate the humid reaction condition. The XRD patterns of ZIF-8W_S and ZIF-8W_T24_S_H in FIG. 24A indicate that transformation from ZIF-8 to DP is inevitable when CO₂ and water are supplied to the reaction. However, the degree of transformation varies depending on the position of the water molecules. Assuming that the same amount of water is applied to the CO₂ cycloaddition reaction including ZIF-8W and ZIF-8W_T24, the formation of the active site during transformation from the ZIF-8 structure to DP was insensitive to water molecules present outside ZIF-8.

Therefore, it was shown that Zn and N species were dissociated in ZIF-8W during the reaction, which was related to the catalytic activity and was accompanied by transformation from ZIF-8 to DP. Transformation from ZIF-8 to DP was also observed even for ZIF-8W_T24 after the reaction (FIG. 24A), but ZIF-8W_T24 did not show any noticeable catalytic activity (FIG. 14C). Therefore, in order to understand the effect of water on the transformation of ZIF-8 structure, the external surface of ZIF-8W_S and ZIF-8W_T24_S_H was characterized. The N is and Zn 2p XPS results show that the external surface components of ZIF-8W_T24_S_H are similar to those of DP, but show that they are significantly different from those of ZIF-8W or ZIF-8W_S (FIG. 25). In addition, O 1 s XPS results showed that ZIF-8W_T24_S_H had additional O species at up to 533.4 eV, and this was consistent with DP (FIG. 24B). Therefore, according to the XPS analysis, although the transformation degree of ZIF-8 structure in ZIF-8W_T_S_H was lower, the external surface of ZIF-8W_T_S_H was similar to that of DP (FIGS. 24A and 24B, and FIG. 25). More importantly, Zn species linked to hydroxyl groups appearing in ZIF-8W_S were absent in ZIF-8W_T24_S_H (FIG. 25B). Therefore, if ZIF-8W_T24_S_H has an external surface composition similar to that of DP, it can be seen that the transformation of ZIF-8W_T24 started from the external surface and progressed (FIGS. 24A and 24B).

Further, as shown in FIGS. 24C1 to 24C2, and 24D, comparison of N₂ physical adsorption and NH₃ TPD results of ZIF-8W_S and ZIF-8W_T24_S_H supports the results obtained from the XPS analysis. N₂ adsorption-desorption isotherms show that, despite the transformation of the ZIF-8 structure, ZIF-8W_T24_S_H retains both of adsorption capacity and unique swing-effect with respect to the adsorption behavior of ZIF-8 in contrast to ZIF-8W_S (FIG. 24C1). As expected, the micropore volume distribution of ZIF-8W, ZIF-8W_S, and ZIF-8W_T24_S_H in FIG. 24C2 indicates that ZIF-8W_T24 maintains a unique micropore structure despite being transformed into DP after the reaction. In ZIF-8W_T24_S_H, the transformation of the ZIF-8 structure is therefore limited to the external surface. The NH₃ TPD result shows that ZIF-8W_T24_S_H has a desorption pattern similar to that of DP, deviating from the pattern of ZIF-8W_S (FIG. 24D). Considering that DP did not show any catalytic activity in the CO₂ cycloaddition reaction, the formation of a DP-like phase on the external surface of ZIF-8W_T24_S_H did not yield an active component suitable for the CO₂ cycloaddition reaction. Then, the noticeable discrepancy between these ZIF-8W_S and ZIF-8W_T24_S_H could be due to the unstable state of the occluding water molecules. Under reaction conditions, such water molecules want to exit the ZIF-8 pore structure, but do not have enough energy to escape (although the synthesized ZIF-8W has been dried at 70° C. for at least 12 hours, it should be noticed that water (8 wt %) is still contained inside the molecules). Instead, at a reaction temperature of 70° C., hydrolysis of the Zn—N bonds appears to yield the active components, thereby yielding ones on the external surface in the aforementioned ZIF-8 intermediate derivatives. However, when structural transformation to DP is done too much, it is not acceptable to have such derivatives or phases. In the case of ZIF-8W_T24_S_H, hydrolysis via water molecules does not appear to be activated at the reaction temperature.

### 7. Confirmation of Active Site of ZIF-8W in Cycloaddition Reaction Using Catalyst

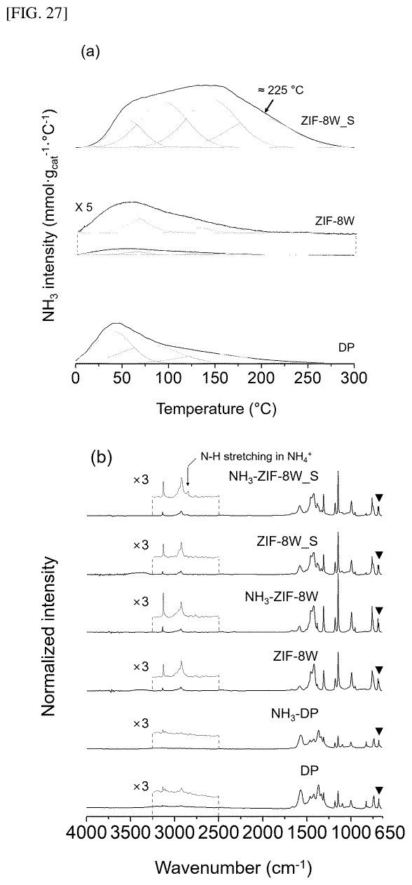

FIG. 26 shows a temperature programmed desorption (TPD) profile for each sample. It shows CO₂ TPD profiles (FIG. 26A) and NH₃ TPD profiles (FIG. 26B) for ZIF-8x and ZIF-8x_S (x=W, M, and C). A tailed arrow in FIG. 26A and FIG. 26B indicates an additional peak appearing in ZIF-8W_S.

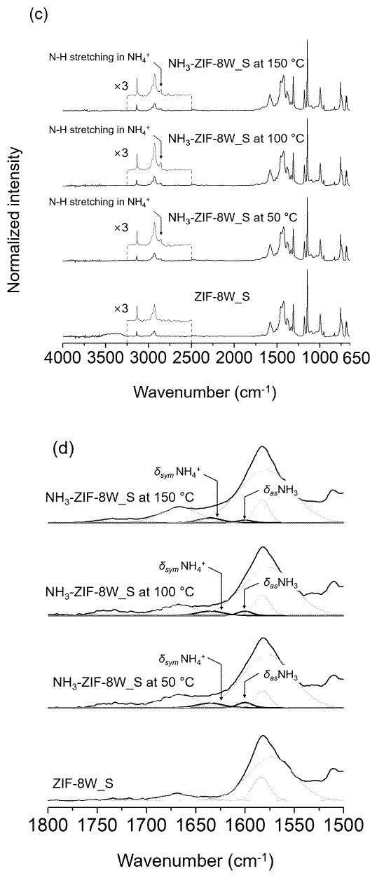

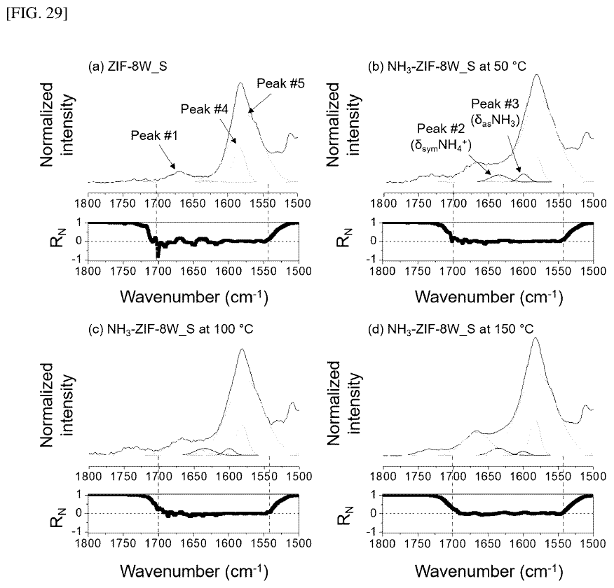

FIG. 27 shows results of performing various types of experiments in order to confirm the active sites in ZIF-8W. FIG. 27A shows NH₃ TPD profiles in ZIF-8W, ZIF-8W_S, and DP. FIG. 27B is FT-IR spectra of ZIF-8W, ZIF-8W_S, and DP before and after NH₃ adsorption (NH₃ is tagged in the corresponding graph). FIG. 27C is FT-IR spectra of NH₃-adsorbed ZIF-8W_S along with the respective tagged desorption temperatures (50, 100, and 150° C.), and FIG. 27D is FT-IR spectra enlarged in the range from 1,800 cm⁻¹ to 1,500 cm⁻¹ along with the deconvoluted curves. In FIGS. 27B to 27D, after performing NH₃ adsorption for FT-IR measurement at 30° C. for 1 hour, the NH₃-adsorbed samples show ones which were heated at different temperatures, respectively, (50° C. in FIG. 27B, and 50, 100 and 150° C. in FIGS. 27C and 27D). All FT-IR spectra were normalized to a peak at 684 cm⁻¹ corresponding to bending of the imidazole ring (indicated by an inverted triangle in FIG. 27B). Detailed information on the deconvolution curve in FIG. 27D is shown in Table 5 and FIG. 29.

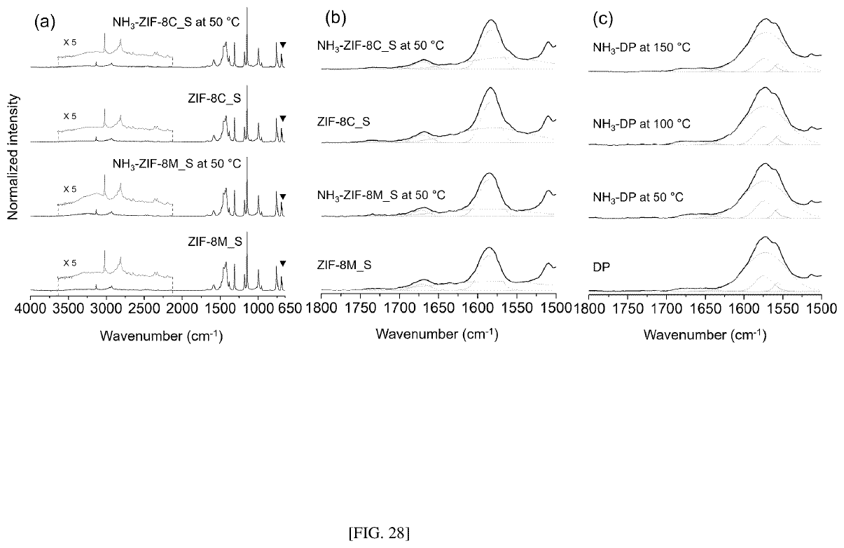

FIG. 28 shows FT-IR spectra before and after NH₃ adsorption. FIG. 28A is FT-IR spectra of ZIF-8M_S and ZIF-8C_S before and after adsorption (NH₃ is tagged on the sample label). FIG. 28B shows enlarged FT-IR spectra of ZIF-8M_S and ZIF-8C_S before and after NH₃ adsorption in the range of 1,800 to 1,500 cm⁻¹. FIG. 28C shows enlarged FT-IR spectra of DP before and after NH₃ adsorption in the range of 1,800 to 1,500 cm⁻¹. After performing NH₃ adsorption at 30° C. for 1 hour for FT-IR measurement, the NH₃-adsorbed ZIF-8M_S, ZIF-8C_S, and DP were heated to the set temperature under He flow. Here, FIGS. 28A and 28B represent ZIF-8M_S and ZIF-8C_S heated to 50° C., respectively, and FIG. 28C represents DP heated to 50, 100 and 150° C. The desorption temperature is indicated at the end of each sample.

FIG. 29 shows information on deconvolution curves. FT-IR spectra of ZIF-8W_S (FIG. 29A) and NH3 adsorbed ZIF-8W_S (FIGS. 29B to 29D) are shown at different temperatures (50, 100, and 150° C.) along with the deconvolved curve, respectively, which are the same as those shown in FIG. 27D. For deconvolution, the region indicated by the dotted line (from 1,540 to 1,700 cm⁻¹) was considered. To demonstrate the appropriate fitting, the FT-IR spectra from which the corresponding residual plots were experimentally obtained are shown below. For residual plots, the normalized residual (R_{N}) was calculated by subtracting the intensity of the experimentally measured FT-IR spectrum from the intensity summed from the deconvoluted curve and further dividing by the intensity of the experimentally measured FT-IR spectrum.

TABLE 5

| | Deconvolution curve (area) | | | | |
|---|---|---|---|---|---|
| | | Curve #2(δ_{sym} | Curve #3(δₐₛ | | |
| | Curve #1 | NH₄⁺) | NH₃) | Curve #4 | Curve #5 |
| Sample | (1,666.6 cm⁻¹) | (1,640.4 cm⁻¹) | (1,600.4 cm⁻¹) | (1,853.4 cm⁻¹) | (1,576.9 cm⁻¹) |
| ZIF-8W_S | 4.37 ± 0.08 | N/A | N/A | 7.13 ± 0.27 | 35.4 ± 0.23 |
| NH₂-ZIF- | 8.75 ± 0.38 | 2.04 ± 0.68 | 1.36 ± 0.39 | 4.63 ± 0.29 | 47.8 ± 1.02 |
| 8W_S at | | | | | |
| 50° C. | | | | | |
| NH₂-ZIF- | 6.77 ± 0.29 | 1.90 ± 0.39 | 1.22 ± 0.24 | 5.79 ± 0.28 | 44.3 ± 0.52 |
| 8W_S at | | | | | |
| 100° C. | | | | | |
| NH₂-ZIF- | 10.3 ± 0.32 | 1.82 ± 0.42 | 0.69 ± 0.31 | 6.06 ± 0.33 | 50.7 ± 0.46 |
| 8W_S at | | | | | |
| 150° C. | | | | | |

Considering that the N and Zn species have similar ratios in ZIF-8x and ZIF-8x_S (x=M and C) (FIGS. 23A1 to 23A3), the only catalytic activity of ZIF-8W is due to newly formed Zn species during the CO₂ cycloaddition reaction. To clarify the role of the newly formed Zn species, CO₂ and NH₃ TPD analysis (FIG. 26) was performed. First of all, the CO₂ TPD results showed that both of ZIF-8W and DP had three main desorption peaks at 30, 60, and 100 to 110° C., whereas ZIF-8W_S had an additional desorption peak at 140° C. (marked with an arrow in FIG. 26A). Referring to the fact that oxygen in the hydroxyl group can act as a Lewis base, oxygen linked to Zn acts as a Lewis base in the CO₂ cycloaddition reaction. In addition, the amount of CO₂ molecules desorbed at about 100° C. was 2.7 μmol·g_{catalyst}⁻¹ in ZIF-8W, but increased to 7.7 μmol·g_{catalyst}⁻¹ in ZIF-8W_S. This corresponded to increased proportions of pyrrolic and pyridinic N species after CO₂ cycloaddition reaction. In contrast, as expected from the distribution of N species in the XPS spectra, the amounts corresponding to desorbed CO₂ for ZIF-8M and ZIF-8C before and after the CO₂ cycloaddition reaction were comparable (FIG. 26A). That is, the amounts of desorbed CO₂ for fresh and spent ZIF-8M at about 100° C. were 1.9 and 1.6 μmol·g_{catalyst}⁻¹, respectively, whereas they were 1.6 and 2.4 μmol·g_{catalyst}⁻¹ at the same temperature for ZIF-8C, respectively. Thus, CO₂ desorption increased at about 100° C. is associated with additional pyrrolic and pyridinic N species. Therefore, the new desorption peak at 140° C. in ZIF-8W_S is due to the connection of oxygen with Zn through a hydroxyl group, and this acts as a Lewis base.

NH₃ TPD results revealed that only ZIF-8W_S had an additional desorption peak at 225° C. after CO₂ cycloaddition reaction (FIG. 27A and FIG. 26B). In addition, the amount of desorbed NH₃ at all desorption peaks was higher than that of ZIF-8W_S. In particular, the amount of NH₃ desorbed from ZIF-8W_S was almost 4 times higher than that of ZIF-8W. NH₃ molecules desorbed from ZIF-8W_S can titrate acidic moieties that may arise from dissociation of Zn—N bonds, but these also arise due to physical adsorption. The amount of NH₃ desorbed from ZIF-8W_S at low temperatures (<100° C.) was much higher compared to ZIF-8W, and such an increase was also seen in DP. The increased desorption in ZIF-8W_S at low temperatures is related to the formation of DP as the final structure instead of forming the desired acidic site in the CO₂ cycloaddition reaction. The additional peak (indicated by an arrow in FIG. 26B) at 225° C. was not clearly understood, but it may be due to the acidic site derived from the dissociation of the Zn—N bonds almost during CO₂ cycloaddition. ZIF-8M and ZIF-8C (FIGS. 21 and 22, and FIG. 5), which preserve the original Zn—N bond after CO₂ cycloaddition reaction, did not show additional peaks in their spent form (FIG. 26B).

The CO₂ cycloaddition reaction takes place at Lewis acid sites, not at Brønsted acidic sites. Since NH₃ TPD results only provide information on all acid sites, it is necessary to examine the acid properties of ZIF-8W_S. Therefore, in order to distinguish the acidic site of ZIF-8W_S, FT-IR measurements of DP, ZIF-8W, and ZIF-8W_S after adsorption of NH₃ were performed, which were interpreted using the NH₃ TPD curve at high temperatures (>100° C.) (FIG. 27A). It is known that the Zn—OH group can act as a Brønsted acidic site, and NH₃ adsorption peak corresponding thereto appears at about 200 to 300° C. Since the Zn—OH group arises from the dissociation of Zn—N during the CO₂ cycloaddition reaction (FIGS. 4B1 and 4B2), the additional peak in the NH₃ TPD result of ZIF-8W_S is due to the Brønsted acidic site. The FT-IR results of NH₃ adsorption shown in FIGS. 27B and 28A show that only ZIF-8W_S has additional FT-IR peaks, which correspond to N—H peaks at ammonium ions (i.e., Brønsted acidic sites; indicated by arrows tagged with ammonium ions in FIG. 27, at 2,850 cm⁻¹). NH₃ desorption in ZIF-8W_S was performed at different temperatures (50, 100, and 150° C.) and FT-IR spectra corresponding thereto show that the FT-IR peak intensities of N—H stretching (2,850 cm⁻¹) were similar regardless of the desorption temperature (FIG. 27C). Therefore, considering that the NH₃ TPD curve of ZIF-8W_S has four main peaks in the middle of about 60, 95, 140, and 225° C. (FIG. 27A), the desorption peak for 150° C. was associated with the Brønsted acidic site, and was apparently due to the formation of Zn—OH groups. The intensity of the broad peak in the FT-IR spectrum ranging from 1,650 cm⁻¹ to 1,600 cm⁻¹ increased after NH₃ adsorption. The FT-IR peaks at 1,645 cm⁻¹ and 1,600 cm⁻¹ correspond to the NH₄⁺ symmetric and NH₃ asymmetric bending modes, respectively. Low-coordinated Zn species behave as Lewis acids, and ZIF-8W_S contains Lewis acidic sites due to Zn species. Based on such a fact, the FT-IR spectra of ZIF-8W_S and NH₃-adsorbed ZIF-8W_S (desorbed at 50, 100, and 150° C.) were deconvoluted using a Gaussian distribution in order to distinguish NH₄⁺ and NH₃ bending modes (FIG. 27D). The deconvolution results showed that the NH₄⁺ symmetric bending modes (1,645 cm⁻¹) representing the Brønsted acidic sites were similar regardless of the desorption temperature. These results were very consistent with the trends in the N—H stretching peaks. In contrast to the NH₄⁺ bending peaks, the NH₃ asymmetric bending modes (at1, 600 cm⁻¹) representing the Lewis acidic sites gradually changed in response to the desorption temperature. The intensities of the NH₃ bending peaks decreased when desorption of NH₃ from ZIF-8W_S was performed at 150° C., whereas the peak intensities at 50° C. and 100° C. were similar (FIG. 27D). According to the XPS analysis, the effective deconvolution of FT-IR shown in FIG. 29 provides tendencies related to the NH₄⁺ and NH₃ bending modes in a quantitative way. Thus, the complementary NH₃ TPD and NH₃ adsorption FT-IR analysis results indicate that the NH₃ TPD desorption at 225° C. is derived from the Brønsted acid site, whereas it is derived from the Lewis acid site in the case of 150° C. In summary, the formation of low-coordinated Zn species is apparently coupled with OH groups, which is provided as Lewis acids in the reaction, and accompanies the structural change of ZIF-8 during the CO₂ cycloaddition reaction.

In contrast to ZIF-8W_S, the FT-IR spectra of NH₃-adsorbed DP show that no acidic sites by NH₃ are found in DP particles regardless of the desorption temperature (FIG. 28C), but the NH₃ TPD desorption peaks existed. Moreover, according to the NH₃ TPD results, ZIF-8M_S and ZIF-8C_S undergoing slight desorption did not show NH₃ adsorption peaks (FIGS. 28A and 28B). In the NH₃ TPD results for ZIF-8W_S and DP, the NH₃ molecules desorbed below 100° C. were related to the structural changes of ZIF-8 (related to the physically adsorbed NH₃ molecules) accompanied during the CO2 cycloaddition reaction, and were not clearly related to the acidic sites.

Those skilled in the art to which the present disclosure pertains will understand that the present disclosure can be embodied in other specific forms without changing its technical spirit or essential features. Therefore, the embodiments described above should be understood as illustrative in all respects and not limiting. The scope of the present disclosure is indicated by the scope of the claims to be described later rather than the detailed description above, and all changes or modified forms derived from the meaning and scope of the claims and equivalent concepts thereof should be interpreted to be included in the scope of the present disclosure.

1. A ZIF-8 metal-organic framework catalyst composite comprising:
a ZIF-8 metal-organic framework in a metal-organic framework; and
water molecules provided inside the ZIF-8 metal-organic framework.

2. The ZIF-8 metal-organic framework catalyst composite of claim 1, wherein the water molecules dissociate at least a portion of the bond of the ZIF-8 metal-organic framework to form a reaction site, and the ZIF-8 metal-organic framework catalyst composite includes a ZIF-8 metal-organic framework in which a reaction site is formed and a ZIF-8 metal-organic framework in which a reaction site is not formed.

3. The ZIF-8 metal-organic framework catalyst composite of claim 2, wherein the reaction site includes:
a first reaction site including a Zn—OH bond; and
a second reaction site including an N—H bond.

4. The ZIF-8 metal-organic framework catalyst composite of claim 2, wherein the formation ratio of the ZIF-8 metal-organic framework in which a reaction site is formed is formed at a weight ratio of 0.05 to 1.0 as the ZIF-8 metal-organic framework in which a reaction site is formed/the ZIF-8 metal-organic framework in which a reaction site is not formed.

5. The ZIF-8 metal-organic framework catalyst composite of claim 3, wherein at least one of the first reaction site and the second reaction site forms carbonate by promoting a cycloaddition reaction between carbon dioxide and an epoxide-based compound.

6. The ZIF-8 metal-organic framework catalyst composite of claim 5, wherein the epoxide-based compound is at least one of epichlorohydrin, ethylene oxide, styrene oxide, and propylene oxide.

7. The ZIF-8 metal-organic framework catalyst composite of claim 5, wherein the yield of carbonate increases as the content of water molecules increases.

8. The ZIF-8 metal-organic framework catalyst composite of claim 5, wherein carbonate is a cyclic carbonate, and is at least one of chloropropene carbonate, ethylene carbonate, styrene carbonate, and propylene carbonate.

9. The ZIF-8 metal-organic framework catalyst composite of claim 5, wherein the yield of carbonate is calculated by Equation 2 below:
$$Yield of carbonate ( \% ) = \frac{Number of moles of formed carbonate}{Number of moles of supplied epoxide - based compound} \times 100. \qquad [ Equation 2 ]$$

10. The ZIF-8 metal-organic framework catalyst composite of claim 9, wherein the yield of carbonate by the cycloaddition reaction is 20% to 99%.

11. The ZIF-8 metal-organic framework catalyst composite of claim 1, wherein the water molecules are contained at a weight ratio of 0.005 to 0.35 with respect to the ZIF-based metal-organic framework catalyst composite.

12. The ZIF-8 metal-organic framework catalyst composite of claim 1, wherein the BET surface area is 1,300 m²/g to 1,600 m²/g.

13. The ZIF-8 metal-organic framework catalyst composite of claim 3, wherein the ZIF-8 metal-organic framework in which a reaction site is formed has a peak for binding energy indicating the formation of Zn—OH in X-ray photoelectron spectroscopy (XPS) analysis, representing a first peak at 1,022.3 eV to 1,022.5 eV and a second peak at 531.8 eV to 520 eV.

14. The ZIF-8 metal-organic framework catalyst composite of claim 3, wherein in X-ray photoelectron spectroscopy (XPS) analysis, the ratio of an XPS peak area of the ZIF-8 metal-organic framework in which a reaction site is formed to that of the ZIF-8 metal-organic framework representing the binding energy of Zn—N is 0.4 to 0.8.

15. A method for converting carbon dioxide using a ZIF-8 metal-organic framework catalyst composite, as a method for converting carbon dioxide using the ZIF-8 metal-organic framework catalyst composite according to claim 1, the method comprising the steps of:
putting a material containing the ZIF-8 metal-organic framework catalyst composite and an epoxide-based compound into an autoclave;
supplying carbon dioxide into the autoclave;
heating the inside of the autoclave to obtain carbonate by reacting the ZIF-8 metal-organic framework catalyst composite, the epoxide-based compound, and carbon dioxide; and
quenching the product.

16. The method of claim 15, wherein the inside of the autoclave is heated to 40° C. to 200° C.

17. The method of claim 15, wherein carbon dioxide is supplied until it reaches a pressure of 1 bar to 30 bar.

18. The method of claim 15, wherein the yield of carbonate is calculated by Equation 2 below:
$$Yield of carbonate ( \% ) = \frac{Number of moles of formed carbonate}{Number of moles of supplied epoxide - based compound} \times 100. \qquad [ Equation 2 ]$$

19. The method of claim 18, wherein the yield of carbonate is 20% to 99%.
